SEQUENCE LISTING (20 sequences)

SEQ ID NO: 1 - protein, 547 aa, Alicyclobacillus ferrooxydans
MDETNFTSTNGSPQYIPVTGSARAIVPGATHAGHTDDNEVLSVTLQLRRPSADELTAHVEALGTTPPANRKHMTHDEFEASHGASDDDLNLVTAFATEQGLSVERINKAAATVHVSGTAGAFNKAFHVQLGNYQHPDFTYRGYDGPVHIPAHLTDIVTGVLGLDNRPQAKPHFRVYQEAAVRSNALAAPISYTPTQVAALYNFPTNVDCSGQCIGIIELGGGYSKSNLDQYFASLGVPTPTITSVSVDGGQNQPTGSPNGPDGEVDLDIEVAASVAPGAHIAVYFAPNTDAGFLDAITTAVHDKTNKPSVISISWGGPEMSWTTQAMQAMNNAMQSAAALGVTITVAAGDNGSTDGVNDGSFHVDFPASAPYALACGGTHLVGSGSTIESETVWNDGANGGATGGGVSSVFPVPSWQQKANVPPSANPGAGTGRGVPDVSGDADPATGYQVLVDGQQFPIGGTSAVAPLWAGLVALANQTLGKPVGYINPLLYSIPAQDNAFHDITQGNNDPNQTGQVYPAGPGWDACTGLGSPNGTLLIQALGQIG

SEQ ID NO: 2 - protein, 524 aa, Caballeronia humi
MSKHPLMGSERAPFDGAQSVGKADPAERLEVTVLVRRGSSDALRTRVSKLVAGNASDGHIQREDFAQQFGAAPNDMSAVRNFASQHGLSVVEEHAARRTVILSGTVAQFNDAFDVDLQQFEHAGGSYRGRTGPVHLPDELSGVVDAVLGLDNRPQARPHFRSRPPQGNVHWQSSRTGTTSSTPLQLASLYDFPAGTGQGQCIAIIELGGGYRPADLKAYFSKLGIASPKVTTVSVDHGKNHPTGDANGPDGEVMLDIEIAGAIAPGAHIAVYFAPNTDAGFLDAVTTAIHDTIRKPSVISISWGGPESAWTEQAMTAFDQAFQAAAALGITVCVASGDNGSGDGVNDGADHVDFPASSPYALACGGTSVQAGKGAIAKETVWNDGANGGASGGGVSSFFALPAWQEGLQAARAKGGTGALQMRGVPDVAGNADPATGYDVRVDGSDMVIGGTSAVAPLWAGLVARINAGKNSPAGYLNPKLYKTAAGLTDITQGNNGDFVASAGWDACTGLGRPDGNKLAGTFG

SEQ ID NO: 3 - protein, 537 aa, Hymenobacter psoromatis
MPQSQNRVVVRGSERQPMPKAHSQHALPPTERLEVTVRLRPKAALASAAASSHAMADVPPSQRTYLSREELAAQCGASEDDAQAVADFAHAHGLVVIHTDLARRSVLLAGTAADFGAAFGTQLHQYSSPEGTYRGRTGTVTVPAPLADIVQGVFGLDDRRQAEPHFQVRPGPTPAPGAIVARAAGQSFTPPQLAQLYDFPGGLDGTGQTIAVIELGGGFKPADLKAYFTGLNLPVPTVKVVSVNGGRNQPTNANSADGEVLLDIEVAAAVAPRAHLVVYFAPNTSQGFLNAITTAVHDKVNNPGIISISWGGPESTWTGQAMDQFDQAFQEAAMLGVTVCVAAGDNGSADGVADGQPHADFPASSPFALACGGTKLTASGPTISSEVVWNEGPNSATGGGLSAHFPVPAYQQQLKFPTPPAGAKAGRGLPDVAGDADPNTGYQVRVDGQNLVIGGTSAVAPLWAGLLALLNQKLPKPVGFLNPLLYGSLAGQGVTRDITSGNNGAFAAGPGWDACTGWGSPVGGKLLAALQGGAAVA

SEQ ID NO: 4 - protein, 539 aa, Caballeronia jiangsuensis
MESIMPSQPSSIPVRGSERAALPTAHVVGPAASDERLEVTLRVRPRAQLHASASEAQSLRPPGERSYLSREQLASAHGAAPEDIAKVEAFAQSHGLQVVLTSAARRCVIVSGTVAALESAFAVKLQQYRFDGGSYRGRVGPVFVSPEIGDIVEGVFGLDDRPQAIAHFKRSAHAVRAEDGAAPHAGGASFTPPQLAKLYNYPGDTDGTGQCIGIVEFGGAIRAADIRAYFKELGLPAPHVNTVLVDHAHMRSDDADAEVMLDIEVAAAIAPKAQIVVYFAPNTSQGFIDAFTHAIHDTVHKPSVISVSWGGPEKDWSAQIKTQLDQVFQDAAALGVTICAASGDAGSSDENPDALASIGLTPDGLSHADFPASSPFALACGGTKLVASASAITSETVWNEDPVRSATGGGISDFFDVPGYQATANIPVSANPGGRKGRGVPDIAADADPATGYLVRVHGQDAVIGGTSAVAPLMAGLVALLNHKLGHPVGFLNPLLYRTAGITRDITQGNNGAYAAGKGWDACTGLGVPDGAKLLDALM

SEQ ID NO: 5 - protein, 549 aa, Terriglobus roseus
MSTRAARTTPSALADLRNEPRSPLPGSEKAALADTPATTAAGIKPLRATAVAKAKPASSRKKITVSVVVPRTKPVTQAAVAGKHLTRAQFKSSHAAAPASVKAVQKFAKAFNLVSKAEPARSTVHLTGTVKDMQDAFGVTLQEHTVGAKTLRIRQGAIYLPDSVLPHVQAVLGLDNRPQAKPHYRVGKARAAASTSFTPPQLAQLYGFPTSAKATGQTIALIELGGGFRQADITAYFKSLGIAAPSVKAVLVDGGKNAPSNANGADGEVMLDIEVAAAVAPGANIAVYFAPNTDQGFVDAIATAAHDTTNKPTIISISWGGPESSWTSQALTALDNACKDAAALGITVTAAAGDDGSDDGVGDGKKHVDFPASSPNVLACGGTKLVASNGAITSEVVWNETANKEGATGGGISTAFPQPTWQKSIAATKSGRGVPDVAGDADPTTGYQVRVDGQNMVIGGTSAVAPLWAGLIALSNATNKNAAGLPQAKLYSTTGQKAFRDITSGNNGAFKAAKGWDPCTGLGSPKAASIITLLATKSSAKKKTSRAKA

SEQ ID NO: 6 - protein, 512 aa, Arthrobacter woluwensis
MAGVNEPYNAREDGIPLKSSARAVVPGVKLHGPTDGASRLEITVVLRRRTELPSAAADGHLTAAELASEYGASDDDVRLATEVFTRLGADVVESDPASRRLRLSGTVEQLSSIFGTTLEDATSTAPDGATVHYRHRLGELRIPAELNGIVIAVLGLDDRPQARAHFRMLPRTTAGTSYSPVELGRVYGFPDGTDGSGQTVAIIELGGGYAQADLDAYFAGLGLATPQITSIGVDGGANQGGNDPQGADGEVLLDIEVVGALAPKAAIQVYFAPNTDAGFLDAVVAATKAAPCAISISWGQSEDQWTAQARDAFDQALADAAALGITTTVAAGDRGSSDGAADGKAHVDFPASSPHALACGGTRLEADPATGAIRSETVWNEGPDSATGGGYSKVFPRPSWQSPSAGKSGRGVPDVSAVADPQTGYRIRVDGKDMVIGGTSAVAPLWAALIARFAQAGNRRFGLIQPSLYAVSSGFRDVTVGDNGSYHAGPGWDACTGLGTPDGAALLAALKG

SEQ ID NO: 7 - protein, 552 aa, Dyella japonica
MSTTAARRHHWPLANEARYLGPHEGTEPMDVTIVMRHRGGRHPEPATWPHQPALPRADFGTQWGADPADADVIRKFASEHGLTEISCALNRRVMHLRGTPQQLQQAFGVAFGSYQMPDGGPAMIGCHLPPSLPSTISPSVIAVLGLDKRPIARPHFRRAATPPATTYTPVQLGSLYQFPANLDGTGQTVAVIELGGGFTQTDFTSYMQSLGLKVPAVNVVSVQGGTSQPGGDADGEVMLDVEVIGALAPGAAIALYFAPNTDQGFYEAISQAAHDAANHPSVISISWGGPEDNWSASSLSAMQSALQDAAAMGVTVTVACGDSGYTDGESDNNPHVDFPASSPYALACGGTTLHGAGTAITSEVVWNDLSTNEGATGGGVSASFALPTWQQSSKVPAGANGYVGRGLPDVAGDADPNTGYQVLVDGQSQVIGGTSAVAPLWAALIARFNQQLGQSVGDPHAALYGIGESAFNDITQGNNGKFNATAGWDACTGLGTPKGQTLLDALAALKGGGGTTAPSPPTNPTSPSKPTKPTKPKKPTKPTKPSKPKPSK

SEQ ID NO: 8 - protein, 528 aa, Bradyrhizobium canariense
MANRKMFPNSVIAIPTSGVTAHGLIVSAADPQSRDEKMDVSFSLGIPPALEKELEERVDKGETIPPQELTTKYAVDPTAAGTLQTWLKKEGFTITGVTPDRTTIYASAPASQVEASLGVHTVRVTREGQTYTAASDVPSLPEDIGGAVVNIGGLQPYRQARKHLRSYIQTTPEADGEEPAIANAPPYLVPEILKAYDGARLGLTGKGQEIAILIDTVPLDTDLTSFWTANGVAGSLARITKINVKGGALPTPSGEETLDAEWASTIAPDANVRIYASGTLSFIDLDRALDRIYADALAQPKLRIVSISLGLSEAYMAKGEVDAEEARFVRFAALGVNVFVSTGDAGSNPGPDGHHANGPLAAEWMSTSPHVVAVGGTSLRLANNGQVASETGWTGSGGGKSNFQPRPAWQQGHGVPAGNQRMVPDVGAAADPNEGALVILNGQRLQYGGTSWSAPIWAGLCALINEARQNNHKTPLPYLNSLIYPMIGSNCFRDELTGSNGAYSCGPGYDLVTGIGSPDLKQLAAKLA

SEQ ID NO: 9 - protein, 595 aa, Burkholderia singularis
MLRNERKIQSGFALGRMAAALVLTIGVHGVAMAVQTQNIGASSWIATRTHAVNTAGATFNGNATDQEPVSIAIALKLRNENALNNYIHELFRRGSPSYHHFLSAKDSVATYAPTQQQAQAVADYLTRQGFTHVRIAPNRLVVSADGTVGAARNAFRTEIAHFTRAGHSGIANTTDIQIPASLSGQVDHVTGLQTLNRMQTFSLQHPSYTTLNSGAAAYAPQEFATVYHAGNTPAGTGTTVALIGWGNMTNPVNDLVQMEKSQNLAAVPTSIEPTSTDSSNDDSTQVEWGMDAQAIVGISGGVKELIFYTSGGNYVQDPKTKSWYSEGSYNSELLKAINLAVSDNKAKVINMSWGLAECRGDAGWADSAFKLGVTQGQTFVAATGDNGAYPCEDSNHHAPANGSYSSNTELSVNYPASSPYVVAVGGTTLNTSASDVYNSEASWPYSGGGISGEESTPTWQPSSYAHRALPDLAFDADSTNSPILIYLTQSQTANISQSGFGYAYGGTSLAAPLFVGAWARLETANKNGMGFAAPAIYSYSTTFPFHDVTTGNNGYYSAAAGWDSATGWGSFDIQAVRDFIANTPGFVSATNAPPN

SEQ ID NO: 10 - protein, 599 aa, Burkholderia singularis
MKKTTSRVFKQGRLAAALAIAFAASGAALADATPGTDASNWVPTRNQVHANATNAQLVGTANDSEPVSITVTMKLRNESKLDQFIKEVHRPGSASFHKFLSASESTADYAPTQQEAQAVADHLTRAGFTNVQIAPNRMAVTAQGTVGAARAAFHTSIGHFNVHGHDAIANTAQVEVPAALSGTVDRVLGLQTLHRPQVFSRTPKPAYTVNSSGGHAYYPKEFATVYNAGSVPAATSTDVAIVGWGTMTNSAADLAYMTKDQGLPSIPTTIKGYGGPTAGATGNDDSGRGEWSMDAQAIAGVSGGVKSLTFYTAYSNIDSRHPYGSATNAGIAAAINGAVSDNIAKVINMSWGDTNSEVCSGTSDTAWLDSYFKLGVAQGQTFSVSSGDNGAYPCNAPQNGAYGDKTQPTVSYPATSPYVVSVGGTTLNTTTTDSYISETVWPYSGGGLSKIEAKPAWQTTVSGNYRAVPDMVFDADWNNSPIRYYMAYVPDSSTSKGGGQTPTQSGYYDNGGTSLASPLFVGSWARLESAHGNSMGFAAPALYAYGTTMPVHDVTSGSNGLYTAGASWDNASGWGSFDISGMSTFITNTPGFVTTSNQH

SEQ ID NO: 11 - protein, 323 aa, Clostridium tetani
MSVRTDLAVEAKEIYEEKNAGEIPGVELKEYRQGRIKVTEVNVLNEQGEKAMNKAIGNYITLEIPDINQYDTQYKEHISKILAKTLMPLLKIDDSMTALVVGLGNWNITPDALGPKVIEKIMITRHLKEYIPNEIDEGIRPVCGISPGVLGITGIETAEIIKAVSNKIKPDIILCIDALASRRLERVNRTIQIGNTGISPGAGVGNRRMELNEKTLGVPVIAIGVPTVVDAATVANDTIDMVLDEMIKVADKDKNFYNMLKSLDRDEKERMIKEVLNPYVGELMVTPKDVDTLMDSISKIISTGINIALQPALELEDINSYLN

SEQ ID NO: 12 - protein, 544 aa, Variovorax sp.
MANGKSTSPASQWVPLPGSNRQLLPQSVPIGPADLKATVALTVKVRSRGKLAELDDAVKKESAKPLKERTYISREELAQRYGADADDLDKVELYANKHHLRVADRDEATRRVVLKGTLEDALSAFHADVHMYQHASGPYRGRRGEILVPAELKDVVTGIFGFDTHPKHRAPRRLMGTSSGTATNLGEFASEFATRYQFPTSSSSTKLDGTGQCIALIELGGGYSNNDLKIFFSEAGVPMPKVVAVSIDHGANHPTPQGLADGEVMLDIEVAGVVAPGAKLAVYFAPNSDSGFQDAIRAAVHDGARKPSVVSISWGEPDDFLTAQSVQSYHEIFTEAAALGVTVCAASGDHGVADLDALHWDKRIHVNHPSSDPLVLCCGGTQIDKNVDVVWNDGTPFDPQVFGGGGWASGGGISPVFGVPDYQKGLPMPSSLSTSQPGRGCPDIAMTADNYRTRVHGVDGPSGGTSAVTPLMACLVARLNQAFEKNLGFVNPLLYANAQAFTDITQGTNGINQTIEGYPAGKGWDACTGLGAPIGTVLLQALGK

SEQ ID NO: 13 - protein, 588 aa, Pseudomonas sp.
MMKSSAAKQTVLCLNRYAVVALPLAIASFAAFGASPASTLWAPTDTKAFVTPAQVEARSAAPLLELAAGETAHIVVSLKLRDEAQLKQLAQAVNQPGNAQFGKFLKRRQFLSQFAPTEAQVQAVVAHLRKNGFVNIHVVPNRLLISADGSAGAVKAAFNTPLVRYQLNGKAGYANTAPAQVPQDLGEIVGSVLGLQNVTRAHPMLKVGERSAAKTLAAGTAKGHNPTEFPTIYDASSAPTAANTTVGIITIGGVSQTLQDLQQFTSANGLASVNTQTIQTGSSNGDYSDDQQGQGEWDLDSQSIVGSAGGAVQQLLFYMADQSASGNTGLTQAFNQAVSDNVAKVINVSLGWCEADANADGTLQAEDRIFATAAAQGQTFSVSSGDEGVYECNNRGYPDGSTYSVSWPASSPNVIAVGGTTLYTTSAGAYSNETVWNEGLDSNGKLWATGGGYSVYESKPSWQSVVSGTPGRRLLPDISFDAAQGTGALIYNYGQLQQIGGTSLASPIFVGLWARLQSANSNSLGFPAASFYSAISSTPSLVHDVKSGNNGYGGYGYNAGTGWDYPTGWGSLDIAKLSAYIRSNGFGH

SEQ ID NO: 14 - protein, 552 aa, Bacillus sp.
MSDMEKPWKEEEKREVLAGHARRQAPQAVDKGPVTGDQRISVTVVLRRQRGDELEAHVERQAALAPHARVHLEREAFAASHGASLDDFAEIRKFAEAHGLTLDRAHVAAGTAVLSGPVDAVNQAFGVELRHFDHPDGSYRSYVGDVRVPASIAPLIEAVLGLDTRPVARPHFRLRRRAEGEFEARSQSAAPTAYTPLDVAQAYQFPEGLDGQGQCIAIIELGGGYDETSLAQYFASLGVSAPQVVSVSVDGATNQPTGDPNGPDGEVELDIEVAGALAPGAKIAVYFAPNTDAGFLNAITTAVHDPTHKPSIVSISWGGPEDSWAPASIAAMNRAFLDAAALGVTVLAAAGDSGSTDGEQDGLYHVDFPAASPYVLACGGTRLVASAGRIERETVWNDGPDGGSTGGGVSRIFPLPSWQERANVPPSANPGAGSGRGVPDVAGNADPATGYEVVIDGETTVIGGTSAVAPLFAALVARINQKLGKPVGYLNPTLYQLPPEVFHDITEGNNDIANRARIYQAGPGWDPCTGLGSPIGIRLLQALLPSASQAQP

SEQ ID NO: 15 - protein, 553 aa, Fimbriimonas sp.
MRHRFGLSILFLVLVSSAVAQVIVPPTSVRRPGERPGTAHTNYRIYIGPWRFPSVDSPFPELAAAHGPAAGQTIPGYHPADIRAAYNVPPNLGTQAIAIVDAFDLPTSLNDFNFFSAQFGLPTEPSGVATASTNRVFQVVYASGTKPATNADWGGEIALDIEWAHAMAPNAKIYLIEADSDSLLDLLAAVRVAATQLSNVRQISMSFGANEFTNESASDSTFLGTNKVFFASSGDASNLVSYPAASPNVVGVGGTRLALSNGSVVSETAWSSAGGGPSSREPRPTYQNSVSGVVGSARGTPDIAAIADPETGVAVYDSTPIPGTGVGWFVVGGTSLACPVCAGITNARGYFTASSFSELTRLYGLAGTSFFRDITSGTSGQFSARVGYDFVTGLGSLLGIFGPFATSPSSLSVVSGTAVAGVPSNMVAKDGHDYVVRSASPAGGGQVATVQGTFASHPPAKAVQFGASVTVTAMRTSGTTTLKLFNQATSAFESVANLTLGTTNTTVTVPIPNAPKYFASDGTTKFQLTTTGPGTTQIRFGVDQVLLTLTPTG

SEQ ID NO: 16 - protein, 514 aa, Pandoraea thiooxydans
MPTFLLPGSEQTCPPGARCVGKADPSARFEVTLVVRQPAQDAFARHLEALHDVTRRPPALTREAYAAQYSAAADDFAAVEQFAASEGLQVVRRDAAQRTIVLSGTVAQFNHAFEIDLQKIEHEGKSYRGRVGPVHLPQHLKTVVDAVLGLEDLPLARTHFRLQPAARSAAGFTPLELASIYQFPAGAGKGQAIALIELGGGVKTSDLTTYFSQLGVTPPQVTAVSVDQATNSPTGDPNGPDGEVTLDVEITGAIAPEAHIVLYFAPNTEAGFFNAVSAAVHDTTHRPTVISISWGGPEAAWTRQSLDAFDRALQAAAAMGVTVCAASGDSGSSGSPGNGSPQVDFPASSPHVLACGGTRLHASANRRDAESVWNDGAGGGASGGGVSAAFALPSWQEGLQVTAADGTSQALTQRGVPDVAGDASPASGYDVVVDAQATIVGGTSAVAPLWAGLIARLNASLGKPLGYLNPILYQHPGVLNDITQGDNGEFSAAPGWDACTGLGSPNGQKIAGVA

SEQ ID NO: 17 - protein, 532 aa, Burkholderiaceae sp.
MVRHPLRGSERTIPEDARILGDAHPAEQIRALVQLRRPNEAELDVRLSGFVHAHAAGTPSPTPLTREEWAAQFGAATDDIDAVRTFAREHGLQVAEVNVAAATVMLEGSVEQFCRAFDTHLHRVAHGGSEYRGRSGPLRLPESLQDVVVAVLGLDSRPQAAPHFRFVPLPTGSVEPGGIRPARAAPTASYTPVQLAQLYGFPQGDGAGQCIAFVELGGGYREDDLRAYFQEVGMPMPTVTAIPVGQGANRPTGDPSGPDGEVMLDLEVAGAAAPGATLAVYFTVNTDAGFVQAINAAIHDTKLRPSVVSISWGAPESAWTPQAMQAVNAALQSAATMGVTVCAASGDSGSSDGQPDRVDHVDFPASSPYALACGGTSVRASGNRIAEETVWNDGARGGAGGGGVSTVFALPSWQQGLAAQQTGGDSVPLARRGVPDVSADADPLTGYVVRVDGESGVVGGTSAAAPLWAALIARINAIKGRPAGYLHARLYQNPGAFNDIKQGNNGAFAAAPGWDACTGLGSPKGDAIANLF

SEQ ID NO: 18 - protein, 550 aa, Paraburkholderia sacchari
MTRHPVSDSGASNEHPVPAGAQCMGACDPAEHFNVVVIVRRQSERAFRELVERIATGAPGAQPISREQYEQRFSADAADVARVEAFAKTHGLVVVKADRDTRRVVLSGTVQQYNAAFGVDLQRFEHQVGKLKQHFRQPTGPVHLPEDLHEVITAVVGLDSRAKVQPHFRIDSQTPATPPEKASQPGDGVVHAPIRAARAVSRSFTPLQLAELYDFPPGDGKGQCIALIEMGGGYAQSDLDAYFSALGVTRPRVEAVSVDQATNAPSGDPNGPDAEVTLDVEIAGALAPGALIAVYFAPNSEAGFVDAVSAALHDSQRKAAIISISWGAPESIWSQQTLGALNDALQTAVALGVTVCCASGDSGSSDGVTDGADHVDFPASSPYALGCGGTQLTAANGRITRETVWGSGANGATGGGVSATFAVPAWQKGLKVSRGSGAARALALARRGVPDVAADADPATGYEVHIGGMDTVVGGTSAVAPLWAALVARINAGSGKAAGFINAKLYARPGAFNDITSGSNGDYAARPGWDACTGLGTPVGTRVAAAIGSA

SEQ ID NO: 19 - protein, 531 aa, Modestobacter sp.
MADDSSPTTAADRPTLPGSARRPVAAAQAAGPLDDAAPLEVTLVLRRRTALPAGTGRPAPMGRAEFAETHGADPADAETVTAALTAEGLRITAVDLPSRRVQVAGDVATFSRVFGVSLSRVESPDPVADRLVPHRQRSGDLAVPAPLAGVVTAVLGLDDRPQARALFRPAAAVDTTFTPLELGRVYRFPSGTDGRGQRLAILELGGGYTQADLDAYWTTIGLADPPTVTAVGVDGAANAPEGDPNGADGEVLLDIEVAGALAPGADLVVYFAPNTDRGFLDALSTAVHADPTPTAVSISWGQNEDEWTAQARTAMDEALADAAALGVTVCAAAGDDGSTDNAPDGQAHVDFPASSPHALACGGTTLRADPDTGEVSSETVWFHGTGQGGTGGGVSAVFAVPDWQDGVRVPGDADTGRHGRGVPDVSADADPSTGYQVRVDGTDAVFGGTSAVSPLWSALTCRLAEALGQRPGLLQPLIYAGLSAGEVAAGFRDVTSGSNGAYDAGPGWDPCTGLGVPDGEALLVRLRTALG

SEQ ID NO: 20 - protein, 515 aa, Pseudonocardia sp.
MSEPVPAAARRTIPGSERPPVDTAAAARQAVPADTRVEATVVLRRRAELPDGPGLLTPAELAERHGADPADVELVTRTLTGLGVEVTAVDAASRRLRVAGPAGVLAEAFGTSLAQVSTPDPSGAQVTHRYRAGALSVPAELDGVVTAVLGLDDRPQARARFRVATAAAASAGYTPIELGRVYSFPEGSDGSGQTIAIIELGGGFAQSELDTYFAGLGISGPTVTAVGVDGGSNVAGRDPQGADGEVLLDIEVAGALAPGADVVVYFAPNTDAGFLDAVAQAAHATPTPAAISISWGGSEDTWTGQARTAFDAALADAAALGVTTTVAAGDDGSTDRATDGKSHVDFPASSPHALACGGTHLDANATTGAVTSEVVWNNGAGKGATGGGVSTVFAQPSWQASAGVPDGPGGKPGRGVPDVSAVADPQTGYRIRVDGQDLVIGGTSAVAPLWAALVARLVQAGRAKLGLLQPKLYAAPTAFRDITEGDNGAYRAGPGWDACTGLGVPVGTALASALS

(19) United States
(12) Patent Application Publication
(10) Pub. No.: US 2026/0139207 A1
(43) Pub. Date: May 21, 2026

(54) WINE DEHAZING METHODS AND COMPOSITIONS

(71) Applicants: DIGESTIVA, INC., Davis, CA (US); VINZYMES, INC., Davis, CA (US)

(72) Inventors: Wai Shun MAK, Sacramento, CA (US); Youtian CUI, Fremont, CA (US); James Austin KENNEDY, Corvallis, OR (US)

(21) Appl. No.: 19/384,727

(22) Filed: Nov. 10, 2025

Related U.S. Application Data
(63) Continuation of application No. PCT/US2024/028933, filed on May 10, 2024.
(60) Provisional application No. 63/501,481, filed on May 11, 2023.

(51) Int. Cl. C12H 1/00; C12G 1/00

(52) U.S. Cl. CPC C12H 1/003; C12G 1/00; C12G 2200/15

(57) ABSTRACT

Provided and described herein are methods and compositions for dehazing wine or another beverage of interest with a protease from the S53 family.

### CROSS-REFERENCE TO RELATED APPLICATION

This application is a National Stage entry of International Application No. PCT/US2024/028933, filed May 10, 2024, which claims the benefit of U.S. Provisional Patent Application No. 63/501,481 filed May 11, 2023.

### SEQUENCE LISTING

Pursuant to the EFS-Web legal framework and 37 C.F.R. § 1.831-835 (see M.P.E.P. § 2413.03), a Sequence Listing in XML format (entitled “3000203-003000_Sequence_Listing” created on 30 Jan. 2026 and 28,899 bytes in size) is submitted concurrently with the instant application, and the entire contents of the Sequence Listing are incorporated herein by reference.

### BACKGROUND

### Field

The formation of haze or precipitates in wine after it is bottled is considered to be a wine defect as it leads to consumer rejection. The most common cause of haze or precipitate formation is attributed to the instability of grape-derived proteins when the wine is exposed to high temperatures and/or interaction of the of grape-derived proteins with tannins. In white wine, this phenomenon is referred to as heat induced protein haze formation or precipitation and, in red wine, this phenomenon is referred to as tannin-induced precipitation. The proteins mostly responsible for white wine haze are thaumatin-like proteins (TLPs), chitinases (CHIs), and B-(l,3)glucanases. These proteins are present in grapes and are released during crushing and pressing.

### SUMMARY

The present disclosure provides methods of dehazing wine comprising contacting a protease from the S53 family with the wine, thereby forming a composition comprising the wine and the protease. The disclosure further provides methods of dehazing a beverage of interest comprising adding a protease from the S53 family to the beverage of interest, thereby forming a composition comprising the beverage of interest and the protease.

An aspect of the present disclosure is a method of dehazing a beverage of interest. The method comprising a step of contacting a protease from the S53 family with the beverage of interest, thereby forming a composition comprising the beverage of interest and the protease.

Another aspect of the present disclosure is a method of dehazing a beverage of interest. The method comprising a step of adding a protease from the S53 family to the beverage of interest, thereby forming a composition comprising the beverage of interest and the protease, wherein the beverage of interest is wine.

In embodiments, the protease degrades heat-unstable proteins causing haze formation in the wine. In various embodiments, the contacting or the adding of the protease to the wine results in a reduced optical density and/or increased clarity in the composition subsequent to the contacting or the adding of the protease relative to an optical density and/or clarity of the wine prior to the contacting or the adding of the protease. In numerous embodiments, the contacting or the adding of the protease to the beverage of interest results in a reduced optical density and/or increased clarity in the composition subsequent to the contacting or the adding of the protease relative to an optical density and/or clarity of the beverage of interest prior to the contacting or the adding of the protease.

In several embodiments, the contacting or the adding of the protease to the beverage of interest results in removal of at least about 85% of heat-unstable proteins in the beverage.

In many embodiments, the contacting or the adding of the protease to the beverage of interest affects the beverage's taste, aroma, color, mouthfeel, and/or perception less than a dehazing method comprising contacting or the adding of bentonite.

In some embodiments, the contacting or the adding of the protease to the beverage of interest does not substantially affect the beverage's taste, aroma, color, mouthfeel, and/or perception.

In some embodiments, the protease is selected from the group consisting of aspartic proteases, cysteine proteases, serine proteases, and metalloproteases.

In embodiments, the S53 family protease comprises or consists of an amino acid sequence having at least 85% sequence identity to any one of SEQ ID NO: 1 to SEQ ID NO: 20. In various embodiments, the protease comprises or consists of a sequence having at least 90% identity to any one of SEQ ID NO: 1 to SEQ ID NO: 20. In numerous embodiments, the S53 family protease comprises or consists of an amino acid sequence having at least 95% sequence identity to any one of SEQ ID NO: 1 to SEQ ID NO: 20. In several embodiments, the S53 family protease comprises or consists of an amino acid sequence having at least 98% sequence identity to any one of SEQ ID NO: 1 to SEQ ID NO: 20. In many embodiments, the protease comprises or consists of a sequence having at least 99% identity to any one of SEQ ID NO: 1 to SEQ ID NO: 20. In some embodiments, the S53 family protease comprises or consists of an amino acid sequence of any one of SEQ ID NO: 1 to SEQ ID NO: 20. In some embodiments, the S53 family protease has an active site comprising one, two, three, four, five, six, seven, eight, nine, ten, eleven, twelve, thirteen, fourteen, fifteen, sixteen, or seventeen residues at position 266, at position 295, at position 316, at position 317, at position 318, at position 349, at position 350, at position 351, at position 352, at position 353, at position 354, at position 367, at position 463, at position 464, at position 465, at position 466, and/or at position 467 each with respect to one of SEQ ID NO: 1 to SEQ ID NO: 20. In embodiments, the S53 family protease comprises an active site wherein at position 266, the amino acid is E or D; at position 295, the amino acid is F, I, or L; at position 316, the amino acid is S; at position 317, the amino acid is W, L, T, or F; at position 318, the amino acid is G; at position 349, the amino acid is A or S; at position 350, the amino acid is A, S, T, or C; at position 351, the amino acid is G; at position 352, the amino acid is D; at position 353, the amino acid is S, N, A, R, E, H, or D; at position 354, the amino acid is G; at position 367, the amino acid is D, S, E, or N; at position 463, the amino acid is G; at position 464, the amino acid is G; at position 465, the amino acid is T; at position 466, the amino acid is S; at position 467, the amino acid is A, L, or W each with respect to one of SEQ ID NO: 1 to SEQ ID NO: 20. In various embodiments, the S53 family protease comprises an active site comprising amino acid residues E266 or D266; F295, 1295, or L295; S316; W317, L317, T317, or F317; G318; A349, S349; A35O, S350, T350, or C350; G351; D352; S353, N353, A353, R353, E353, H353, or D353; G354; D367, S367, E367, or N367; G463; G464; T465; and/or S466; A467, L467, or W467 with respect to any one of SEQ ID NO: 1 to SEQ ID NO: 20. In numerous embodiments, the S53 family protease comprises an active site comprising one or more amino acid substitutions, e.g., one, two, three, four, five, six, seven, eight, nine, ten, eleven, twelve, thirteen, fourteen, fifteen, sixteen, or seventeen substitutions, selected from E266, D266, F295, 1295, L295, S316, W317, L317, T317, F317, G318, A349, S349, A35O, S350, T350, C350, G351, D352, S353, N353, A353, R353, E353, H353, D353, G354, D367, S367, E367, N367, G463, G464, T465, S466, A467, L467, and W467 with respect to any one of SEQ ID NO: 1 to SEQ ID NO: 20. In several embodiments, the S53 family protease comprises or consists of an amino acid sequence having at least 85%, at least 90%, at least 95%, at least 98%, or at least 99% sequence identity to any one of SEQ ID NO: 1 to SEQ ID NO: 20 and comprises an active site comprising one or more amino acid substitutions, e.g., one, two, three, four, five, six, seven, eight, nine, ten, eleven, twelve, thirteen, fourteen, fifteen, sixteen, or seventeen substitutions, selected from E266, D266, F295, 1295, L295, S316, W317, L317, T317, F317, G318, A349, S349, A350, S35O, T350, C350, G351, D352, S353, N353, A353, R353, E353, H353, D353, G354, D367, S367, E367, N367, G463, G464, T465, S466, A467, L467, and W467 with respect to any one of SEQ ID NO: 1 to SEQ ID NO: 20.

In many embodiments, the active site comprises between one and five amino acid substitutions. In embodiments, the active site substitution does not reduce the stability, specificity, and/or activity of the protease. In embodiments, the active site substitution increases the stability, specificity, and/or activity of the protease.

In embodiments, the protease comprises or consists of a sequence having at least 85% identity, 90% identity, 95% identity, 98% identity, 99% identity, or identity to SEQ ID NO: 14. In some embodiments, the S53 family protease comprises an active site comprising one or more amino acid substitutions, e.g., one, two, three, four, five, six, seven, eight, nine, ten, eleven, twelve, thirteen, fourteen, fifteen, sixteen, or seventeen substitutions, selected from E266, D266, F295, 1295, L295, S316, W317, L317, T317, F317, G318, A349, S349, A350, S35O, T350, C350, G351, D352, S353, N353, A353, R353, E353, H353, D353, G354, D367, S367, E367, N367, G463, G464, T465, S466, A467, L467, or W467 of SEQ ID NO: 14. In some embodiments, the S53 family protease comprises or consists of an amino acid sequence having at least 85%, at least 90%, at least 95%, at least 98%, or at least 99% sequence identity to SEQ ID NO: 14 and comprising an active site having one or more amino acid substitutions, e.g., one, two, three, four, five, six, seven, eight, nine, ten, eleven, twelve, thirteen, fourteen, fifteen, sixteen, or seventeen substitutions, selected from E266, D266, F295, 1295, L295, S316, W317, L317, T317, F317, G318, A349, S349, A35O, S350, T350, C350, G351, D352, S353, N353, A353, R353, E353, H353, D353, G354, D367, S367, E367, N367, G463, G464, T465, S466, A467, L467, and W467 of SEQ ID NO: 14. In embodiments, the active site substitution does not reduce the stability, specificity, and/or activity of the protease. In embodiments, the active site substitution increases the stability, specificity, and/or activity of the protease.

In embodiments, the protease comprises or consists of a sequence having at least 85% identity, 90% identity, 95% identity, 98% identity, 99% identity, or identity to SEQ ID NO: 8. In embodiments, the S53 family protease comprises an active site comprising one or more amino acid substitutions, e.g., one, two, three, four, five, six, seven, eight, nine, ten, eleven, twelve, thirteen, fourteen, fifteen, sixteen, or seventeen substitutions, selected from E266, D266, F295, 1295, L295, S316, W317, L317, T317, F317, G318, A349, S349, A350, S35O, T350, C350, G351, D352, S353, N353, A353, R353, E353, H353, D353, G354, D367, S367, E367, N367, G463, G464, T465, S466, A467, E467, or W467 of SEQ ID NO: 8. In various embodiments, the S53 family protease comprises or consists of an amino acid sequence having at least 85%, at least 90%, at least 95%, at least 98%, or at least 99% sequence identity to SEQ ID NO: 8 and comprising an active site having one or more amino acid substitutions, e.g., one, two, three, four, five, six, seven, eight, nine, ten, eleven, twelve, thirteen, fourteen, fifteen, sixteen, or seventeen substitutions, selected from E266, D266, F295, 1295, L295, S316, W317, L317, T317, F317, G318, A349, S349, A35O, S350, T350, C350, G351, D352, S353, N353, A353, R353, E353, H353, D353, G354, D367, S367, E367, N367, G463, G464, T465, S466, A467, L467, and W467 of SEQ ID NO: 8. In embodiments, the active site substitution does not reduce the stability, specificity, and/or activity of the protease. In embodiments, the active site substitution increases the stability, specificity, and/or activity of the protease.

In embodiments, the protease comprises or consists of a sequence having at least 85% identity, 90% identity, 95% identity, 98% identity, 99% identity, or identity to SEQ ID NO: 1. In numerous embodiments, the S53 family protease comprises an active site comprising one or more amino acid substitutions, e.g., one, two, three, four, five, six, seven, eight, nine, ten, eleven, twelve, thirteen, fourteen, fifteen, sixteen, or seventeen substitutions, selected from E266, D266, F295, 1295, L295, S316, W317, L317, T317, F317, G318, A349, S349, A350, S35O, T350, C350, G351, D352, S353, N353, A353, R353, E353, H353, D353, G354, D367, S367, E367, N367, G463, G464, T465, S466, A467, L467, or W467 of SEQ ID NO: 1. In several embodiments, the S53 family protease comprises or consists of an amino acid sequence having at least 85%, at least 90%, at least 95%, at least 98%, or at least 99% sequence identity to SEQ ID NO: 1 and comprising an active site having one or more amino acid substitutions, e.g., one, two, three, four, five, six, seven, eight, nine, ten, eleven, twelve, thirteen, fourteen, fifteen, sixteen, or seventeen substitutions, selected from E266, D266, F295, 1295, L295, S316, W317, L317, T317, F317, G318, A349, S349, A35O, S350, T350, C350, G351, D352, S353, N353, A353, R353, E353, H353, D353, G354, D367, S367, E367, N367, G463, G464, T465, S466, A467, L467, and W467 of SEQ ID NO: 1. In embodiments,

the active site substitution does not reduce the stability, specificity, and/or activity of the protease. In embodiments, the active site substitution increases the stability, specificity, and/or activity of the protease.

In many embodiments, the active site comprises between one and five amino acid substitutions. In embodiments, the amino acid substitution does not reduce the stability, specificity, and/or activity of the protease. In embodiments, the amino acid substitution increases the stability, specificity, and/or activity of the protease.

In some embodiments, the S53 family protease comprises one or more truncations of any one of SEQ ID NO: 1 to SEQ ID NO: 20, wherein the one or more truncations comprises an N-terminal truncation, a C-terminal truncation, or both an N-terminal and C-terminal truncation. In embodiments, a truncation does not reduce the stability, specificity, and/or activity of the protease. In embodiments, a truncation increases the stability, specificity, and/or activity of the protease.

In some embodiments, the S53 family protease comprises one or more truncations of SEQ ID NO: 8, wherein the one or more truncations comprises an N-terminal truncation, a C-terminal truncation, or both an N-terminal and C-terminal truncation. In embodiments, a truncation does not reduce the stability, specificity, and/or activity of the protease. In embodiments, a truncation increases the stability, specificity, and/or activity of the protease.

In embodiments, the S53 family protease comprises one or more truncations of SEQ ID NO: 14, wherein the one or more truncations comprises an N-terminal truncation, a C-terminal truncation, or both an N-terminal and C-terminal truncation. In embodiments, a truncation does not reduce the stability, specificity, and/or activity of the protease. In embodiments, a truncation increases the stability, specificity, and/or activity of the protease.

In various embodiments, the S53 family protease comprises one or more truncations of SEQ ID NO: 1, wherein the one or more truncations comprises an N-terminal truncation, a C-terminal truncation, or both an N-terminal and C-terminal truncation. In embodiments, a truncation does not reduce the stability, specificity, and/or activity of the protease. In embodiments, a truncation increases the stability, specificity, and/or activity of the protease.

In numerous embodiments, a temperature of the composition does not exceed a temperature of 25° C. during performance of the method. In several embodiments, a temperature of the composition does not exceed a temperature of 60° C. during performance of the method. In many embodiments, in a temperature of the composition docs not exceed a temperature of 80° C. during performance of the method.

In some embodiments, a protein load of the protease in the composition does not exceed 1%.

In some embodiments, the composition does not comprise a laccase.

In embodiments, the protease is added to a beverage of interest, e.g., wine, at a concentration ranging from about 0.001 mg/L to about 100 mg/L or from about 0.001 mg/L to about 1000 mg/L, e.g., from about 0.001 mg/L to about 0.01 mg/L; from about 0.01 mg/L to about 0.1 mg/L; from about 0.1 mg/L to about 1 mg/L; from about 1 mg/L to about 10 mg/L; from about 10 mg/L to about 100 mg/L; or from about 100 mg/L to about 1,000 mg/L.

In various embodiments, the beverage of interest is a wine selected from red wine, white wine, and rose wine.

In numerous embodiments, the protease is added to the beverage of interest, e.g., wine at any time prior to bottling.

In several embodiments, the protease is added to a beverage of interest being wine during winemaking.

In many embodiments, the methods comprise incubating the composition for 30 minutes to 72 hours, and any period time therebetween.

In some embodiments, the methods comprise incubating the composition for 3 days to 3 weeks, and any period time therebetween.

In some embodiments, the methods comprise incubating the composition for 3 weeks to 3 months, and any period time therebetween.

In embodiments, the methods comprise incubating the composition for 3 months to 3 years, and any period time therebetween.

In various embodiments, the protease degrades or self-destructs to produce amino acids and/or short-chain polypeptides.

In numerous embodiments, the methods comprise filtering the composition after treatment with the protease to remove residual haze-related precipitates.

In several embodiments, the methods comprise a step of adding a fining agent to the composition, thereby increasing clarity of the composition. In some cases, the fining agent is bentonite. The amount of fining agent used is less than needed to dehaze the beverage of interest in the absence of the protease and/or the amount of protease used is less than needed to dehaze the beverage of interest in the absence of the fining agent. The protease may be added to or contacted with the beverage of interest before adding the fining agent, the protease may be added to or contacted with the beverage of interest after adding the fining agent, or the protease may be added to or contacted with the beverage of interest simultaneous with the adding of the fining agent.

In many embodiments, the protease is immobilized on a solid support prior to contacting with the beverage of interest, e.g., wine. In some cases, the solid support is a column or another device that permits the beverage to flow through and to contact the immobilize protease.

In some embodiments, the methods further comprise monitoring protein content and/or optical density of a sample of the beverage of interest, e.g., wine, and/or the composition to assess the degree of haze reduction.

In some embodiments, the beverage of interest is wine, juice, beer, cider, or any other alcoholic or non-alcoholic beverage containing heat-unstable proteins.

In embodiments, the methods further comprise adding an enzyme other than the protease from the S53 family to the beverage of interest, e.g., wine, or to the composition.

In various embodiments, the juice is grape juice, apple juice, orange juice, berry (e.g., blackberry, blueberry, blackcurrant, redcurrant, cranberry, elderberry, gooseberry, mulberry, seaberry, raspberry, and white-currant) juice, pineapple juice, pomegranate juice, plum juice, dandelion juice, rose hip juice, lychee juice, pear juice, or cherry juice.

In numerous embodiments, the beverage of interest comprises fermented honey.

In several embodiments, the beverage of interest comprises fermented grain, e.g., rice, bailey, corn, rye, or wheat.

A further aspect of the present disclosure is a composition comprising a protease from the S53 family and a liquid.

An additional aspect of the present disclosure is a composition comprising a protease from the S53 family and a beverage of interest, e.g., wine, juice, beer, cider, or any other alcoholic or nonalcoholic beverage containing heat-unstable proteins.

In many embodiments, the S53 family protease in a composition comprises or consists of an amino acid sequence having at least 85% sequence identity to any one of SEQ ID NO: 1 to SEQ ID NO: 20. In some embodiments, the protease in a composition comprises or consists of a sequence having at least 90% identity to any one of SEQ ID NO: 1 to SEQ ID NO: 20. In some embodiments, the S53 family protease in a composition comprises or consists of an amino acid sequence having at least 95% sequence identity to any one of SEQ ID NO: 1 to SEQ ID NO: 20. In embodiments, the S53 family protease in a composition comprises or consists of an amino acid sequence having at least 98% sequence identity to any one of SEQ ID NO: 1 to SEQ ID NO: 20. In various embodiments, the protease in a composition comprises or consists of a sequence having at least 99% identity to any one of SEQ ID NO: 1 to SEQ ID NO: 20. In numerous embodiments, the S53 family protease in a composition comprises or consists of an amino acid sequence of any one of SEQ ID NO: 1 to SEQ ID NO: 20. In several embodiments, the S53 family protease has an active site comprising one, two, three, four, five, six, seven, eight, nine, ten, eleven, twelve, thirteen, fourteen, fifteen, sixteen, or seventeen residues at position 266, at position 295, at position 316, at position 317, at position 318, at position 349, at position 350, at position 351, at position 352, at position 353, at position 354, at position 367, at position 463, at position 464, at position 465, at position 466, and/or at position 467 each with respect to one of SEQ ID NO: 1 to SEQ ID NO: 20. In many embodiments, the S53 family protease comprises an active site wherein at position 266, the amino acid is E or D; at position 295, the amino acid is F, I, or L; at position 316, the amino acid is S; at position 317, the amino acid is W, L, T, or F; at position 318, the amino acid is G; at position 349, the amino acid is A or S; at position 350, the amino acid is A, S, T, or C; at position 351, the amino acid is G; at position 352, the amino acid is D; at position 353, the amino acid is S, N, A, R, E, H, or D; at position 354, the amino acid is G; at position 367, the amino acid is D, S, E, or N; at position 463, the amino acid is G; at position 464, the amino acid is G; at position 465, the amino acid is T; at position 466, the amino acid is S; at position 467, the amino acid is A, L, or W each with respect to one of SEQ ID NO: 1 to SEQ ID NO: 20. In some embodiments, the S53 family protease in a composition comprises an active site comprising amino acid residues E266 or D266; F295, 1295, or L295; S316; W317, L317, T317, or F317; G318; A349, S349; A35O, S350, T350, or C350; G351; D352; S353, N353, A353, R353, E353, H353, or D353; G354; D367, S367, E367, or N367; G463; G464; T465; and/or S466; A467, L467, or W467 with respect to any one of SEQ ID NO: 1 to SEQ ID NO: 20. In some embodiments, the S53 family protease in a composition comprises an active site comprising one or more amino acid substitutions, e.g., one, two, three, four, five, six, seven, eight, nine, ten, eleven, twelve, thirteen, fourteen, fifteen, sixteen, or seventeen substitutions, selected from E266, D266, F295, 1295, L295, S316, W317, L317, T317, F317, G318, A349, S349, A350, S350, T350, C350, G351, D352, S353, N353, A353, R353, E353, H353, D353, G354, D367, S367, E367, N367, G463, G464, T465, S466, A467, L467, and W467 with respect to any one of SEQ ID NO: 1 to SEQ ID NO: 20. In embodiments, the S53 family protease comprises or consists of an amino acid sequence having at least 85%, at least 90%, at least 95%, at least 98%, or at least 99% sequence identity to any one of SEQ ID NO: 1 to SEQ ID NO: 20 and comprising an active site having one or more amino acid substitutions, e.g., one, two, three, four, five, six, seven, eight, nine, ten, eleven, twelve, thirteen, fourteen, fifteen, sixteen, or seventeen substitutions, selected from E266, D266, F295, 1295, L295, S316, W317, L317, T317, F317, G318, A349, S349, A350, S350, T350, C350, G351, D352, S353, N353, A353, R353, E353, H353, D353, G354, D367, S367, E367, N367, G463, G464, T465, S466, A467, L467, and W467 of any one of SEQ ID NO: 1 to SEQ ID NO: 20. In various embodiments, the active site comprises between one and five amino acid substitutions. In embodiments, the active site substitution does not reduce the stability, specificity, and/or activity of the protease. In embodiments, the active site substitution increases the stability, specificity, and/or activity of the protease.

In embodiments, the S53 family protease in a composition comprises or consists of a sequence having at least 85% identity, 90% identity, 95% identity, 98% identity, 99% identity, or identity to SEQ ID NO: 8. In numerous embodiments, the S53 family protease in a composition comprises an active site comprising one or more amino acid substitutions, e.g., one, two, three, four, five, six, seven, eight, nine, ten, eleven, twelve, thirteen, fourteen, fifteen, sixteen, or seventeen substitutions, selected from E266, D266, F295, 1295, L295, S316, W317, L317, T317, F317, G318, A349, S349, A35O, S350, T35O, C350, G351, D352, S353, N353, A353, R353, E353, H353, D353, G354, D367, S367, E367, N367, G463, G464, T465, S466, A467, L467, or W467 of SEQ ID NO: 8. In several embodiments, the S53 family protease comprises or consists of an amino acid sequence having at least 85%, at least 90%, at least 95%, at least 98%, or at least 99% sequence identity to SEQ ID NO: 8 and comprising an active site having one or more amino acid substitutions, e.g., one, two, three, four, five, six, seven, eight, nine, ten, eleven, twelve, thirteen, fourteen, fifteen, sixteen, or seventeen substitutions, selected from E266, D266, F295, 1295, L295, S316, W317, L317, T317, F317, G318, A349, S349, A350, S35O, T350, C350, G351, D352, S353, N353, A353, R353, E353, H353, D353, G354, D367, S367, E367, N367, G463, G464, T465, S466, A467, E467, and W467 of SEQ ID NO: 8. In embodiments, the active site substitution does not reduce the stability, specificity, and/or activity of the protease. In embodiments, the active site substitution increases the stability, specificity, and/or activity of the protease.

In embodiments, the S53 family protease in a composition comprises or consists of a sequence having at least 85% identity, 90% identity, 95% identity, 98% identity, 99% identity, or identity to SEQ ID NO: 14. In many embodiments, the S53 family protease in a composition comprises an active site comprising one or more amino acid substitutions, e.g., one, two, three, four, five, six, seven, eight, nine, ten, eleven, twelve, thirteen, fourteen, fifteen, sixteen, or seventeen substitutions, selected from E266, D266, F295, 1295, L295, S316, W317, L317, T317, F317, G318, A349, S349, A35O, S350, T35O, C350, G351, D352, S353, N353, A353, R353, E353, H353, D353, G354, D367, S367, E367, N367, G463, G464, T465, S466, A467, L467, or W467 of SEQ ID NO: 14. In some embodiments, the S53 family protease comprises or consists of an amino acid sequence having at least 85%, at least 90%, at least 95%, at least 98%, or at least 99% sequence identity to SEQ ID NO: 14 and comprising an active site having one or more amino acid substitutions, e.g., one, two, three, four, five, six, seven, eight, nine, ten, eleven, twelve, thirteen, fourteen, fifteen, sixteen, or seventeen substitutions, selected from E266, D266, F295, 1295, L295, S316, W317, L317, T317, F317, G318, A349, S349, A350, S35O, T350, C350, G351, D352, S353, N353, A353, R353, E353, H353, D353, G354, D367, S367, E367, N367, G463, G464, T465, S466, A467, L467, and W467 of SEQ ID NO: 14. In embodiments, the active site substitution does not reduce the stability, specificity, and/or activity of the protease. In embodiments, the active site substitution increases the stability, specificity, and/or activity of the protease.

In embodiments, the S53 family protease in a composition comprises or consists of a sequence having at least 85% identity, 90% identity, 95% identity, 98% identity, 99% identity, or identity to SEQ ID NO: 1. In some embodiments, the S53 family protease in a composition comprises an active site comprising one or more amino acid substitutions, e.g., one, two, three, four, five, six, seven, eight, nine, ten, eleven, twelve, thirteen, fourteen, fifteen, sixteen, or seventeen substitutions, selected from E266, D266, F295, 1295, L295, S316, W317, L317, T317, F317, G318, A349, S349, A35O, S350, T35O, C350, G351, D352, S353, N353, A353, R353, E353, H353, D353, G354, D367, S367, E367, N367, G463, G464, T465, S466, A467, L467, or W467 of SEQ ID NO: 1. In embodiments, the S53 family protease comprises or consists of an amino acid sequence having at least 85%, at least 90%, at least 95%, at least 98%, or at least 99% sequence identity to SEQ ID NO: 1 and comprising an active site having one or more amino acid substitutions, e.g., one, two, three, four, five, six, seven, eight, nine, ten, eleven, twelve, thirteen, fourteen, fifteen, sixteen, or seventeen substitutions, selected from E266, D266, F295, 1295, L295, S316, W317, L317, T317, F317, G318, A349, S349, A350, S350, T350, C350, G351, D352, S353, N353, A353, R353, E353, H353, D353, G354, D367, S367, E367, N367, G463, G464, T465, S466, A467, L467, and W467 of SEQ ID NO: 1. In embodiments, the active site substitution does not reduce the stability, specificity, and/or activity of the protease. In embodiments, the active site substitution increases the stability, specificity, and/or activity of the protease.

In various embodiments, the active site comprises between one and five amino acid substitutions. In embodiments, the amino acid substitution does not reduce the stability, specificity, and/or activity of the protease. In embodiments, the amino acid substitution increases the stability, specificity, and/or activity of the protease.

In numerous embodiments, the S53 family protease in a composition comprises one or more truncations of any one of SEQ ID NO: 1 to SEQ ID NO: 20, wherein the one or more truncations comprises an N-terminal truncation, a C-terminal truncation, or both an N-terminal and C-terminal truncation. In embodiments, a truncation does not reduce the stability, specificity, and/or activity of the protease. In embodiments, a truncation increases the stability, specificity, and/or activity of the protease.

In several embodiments, the S53 family protease in a composition comprises one or more truncations of SEQ ID NO: 8, wherein the one or more truncations comprises an N-terminal truncation, a C-terminal truncation, or both an N-terminal and C-terminal truncation. In embodiments, a truncation does not reduce the stability, specificity, and/or activity of the protease. In embodiments, a truncation increases the stability, specificity, and/or activity of the protease.

In many embodiments, the S53 family protease in a composition comprises one or more truncations of SEQ ID NO: 14, wherein the one or more truncations comprises an N-terminal truncation, a C-terminal truncation, or both an N-terminal and C-terminal truncation. In embodiments, a truncation does not reduce the stability, specificity, and/or activity of the protease. In embodiments, a truncation increases the stability, specificity, and/or activity of the protease.

In some embodiments, the S53 family protease in a composition comprises one or more truncations of SEQ ID NO: 1, wherein the one or more truncations comprises an N-terminal truncation, a C-terminal truncation, or both an N-terminal and C-terminal truncation. In embodiments, a truncation does not reduce the stability, specificity, and/or activity of the protease. In embodiments, a truncation increases the stability, specificity, and/or activity of the protease.

In some embodiments, the protease is present in the composition at a concentration ranging from about 0.001 mg/L to about 100 mg/L or from about 0.001 mg/L to about 1000 mg/L, e.g., from about 0.001 mg/L to about 0.01 mg/L; from about 0.01 mg/L to about 0.1 mg/L; from about 0.1 mg/L to about 1 mg/L; from about 1 mg/L to about 10 mg/L; from about 10 mg/L to about 100 mg/L; or from about 100 mg/L to about 1,000 mg/L.

In embodiments, the beverage of interest is a wine selected from red wine, white wine, and rose wine.

In various embodiments, the juice is grape juice, apple juice, orange juice, berry (e.g., blackberry, blueberry, blackcurrant, redcurrant, cranberry, elderberry, gooseberry, mulberry, seaberry, raspberry, and white-currant) juice, pineapple juice, pomegranate juice, plum juice, dandelion juice, rose hip juice, lychee juice, pear juice, or cherry juice.

In numerous embodiments, the beverage of interest comprises fermented honey.

In several embodiments, the beverage of interest comprises fermented grain, e.g., rice, barley, corn, rye, or wheat.

In many embodiments, the protease degrades heat-unstable proteins that cause haze formation in the beverage of interest, e.g., wine.

In some embodiments, the protease is added to the wine or the beverage of interest at any time prior to bottling.

In some embodiments, the protease is added to the beverage of interest being wine during winemaking.

In various embodiments, the protease degrades or self-destructs to produce amino acids and/or short-chain polypeptides.

In numerous embodiments, the composition further comprises a fining agent.

In several embodiments, the protease is immobilized on a solid support.

In many embodiments, the beverage of interest is wine, juice, beer, cider, or any other alcoholic or non-alcoholic beverage containing heat-unstable proteins.

In some embodiments, the composition further comprises an enzyme other than the protease from the S53 family.

Yet another aspect of the present disclosure is a solid support comprising any herein-disclosed composition, with the protease being immobilized onto the solid support.

Any composition or method disclosed herein is applicable to any herein-disclosed composition or method. In other words, any aspect or embodiment described herein can be combined with any other aspect or embodiment as disclosed herein.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 is a graph showing the effect of enzyme concentration on haze formation.

FIG. 2 is a graph showing the conversion rate of major heat-induced haze proteins by protease treatment.

FIG. 3 is a graph showing efficacy of S53 proteases in reducing wine haze and turbidity after heat test.

FIG. 4 is a graph showing wine dehazing of illustrative wine varietals by a protease of the present disclosure.

FIG. 5 is a graph showing dehazing by a protease of the present disclosure of juice while fermenting into wine.

FIG. 6 is a graph showing wine dehazing by numerous commercially-available proteases.

### DETAILED DESCRIPTION OF A PREFERRED EMBODIMENT

### Introduction

The wine industry has struggled to find a practical solution to the issue of heat-induced protein haze, which leads to significant economic loss. Various solutions have been proposed, but the most common approach involves removing the heat-unstable proteins by fining with bentonite, which is an absorbent, swelling clay. Indeed, bentonite is uniquely useful in the process of winemaking, where it is used to remove excessive amounts of protein from white wines which would precipitate undesirable flocculent clouds or hazes upon exposure to warm temperatures, as these proteins denature. Bentonite is also uses for rapid clarification of both red and white wines. However, use of bentonite has a number of drawbacks. First, bentonite for wine treatment can cause significant economic loss due to wine volume loss. Second, bentonite fining is time-consuming. Third, bentonite fining leads to wine quality degradation by non-selectively adsorbing desirable wine flavor components and other important components. In addition, bentonite fouls wine filters quickly, and can damage processing equipment due to its abrasive characteristics. Globally, the estimated total economic loss incurred annually due to bentonite-based fining amounts to approximately $1 billion per year'. The use of alternative porous adsorbent materials such as zeolites, grape seed powder, and zirconium oxide are currently being evaluated, but further research is needed to determine their impact on wine quality and safety.

Alternately, additive-stabilizing approaches using animal-based or plant-based agents, such as mannoproteins, carrageenan/pecan, and chitosan, have been proposed to prevent haze formation. However, these techniques have led to unpredictable physical and chemical results and which negatively affect sensory characteristics of the de-hazed wine.

The use of proteolytic enzymes (e.g., peptidases and proteases) have been considered as an alternative to traditional bentonite fining to prevent protein haze formation. Recent research has been focused on providing an enzymatic treatment to prevent haze formation during any stage of winemaking, i.e., from the initial fermentation stage to bottling. However, these preliminary results have shown numerous disadvantages in the winemaking process. As examples, the protease BcAP8. From the grape fungal pathogen Botrytis cinerea, degrades grape-derived chitinases (CHIs), but it did not statistically reduce the levels of other grape-derived proteins: thaumatin-like proteins (TLPs). Use of Aspergillopepsin I and II requires preheating steps of 60-80° C. to function; this heating not only results in a loss of aroma but also increases operating costs.

Although use of Aspartic MpAPrl from Metschnikowia pulcherrima reduces about 50% of CHIs and TLPs when supplemented in the fermentation, for the enzyme to perform, the fermentation must be mainlined at 25° C., which is higher than normal and likely affects the resulting wine product. Proline-specific endopeptidases (Brewers Clarex®) from Aspergillus niger have been reported to reduce protein haze under various conditions but only when in combination with a laccase. Bromelain, which is derived from pineapple stems, only functions when under high enzyme concentrations (e.g., about 1% w/v), which results in high costs. While various enzymes have been considered for de-hazing wine, each has significant drawbacks. In summary, the known challenges from using proteases is that they only function under conditions, e.g., low pH, low alcohol content, and low temperature, which is contrary to winemaking processes. Despite these limitations, enzymatic methods have potential.

In summary, the wine industry has long struggled with the issue of heat induced protein haze formation and the currently-most used solution to this issue which is bentonite-based and the proposed enzymatic solutions have a number of drawbacks, as discussed above. These drawbacks are avoided by the methods and compositions of the present disclosure.

The present disclosure provides new methods for preventing or reducing haze in wine caused by heat-induced protein precipitation which provide several advantages over previously-explored dehazing methods. These methods involve the use of acid-active proteases from the S53 family. These proteases do not require a heating step, which could harm wine quality, yet provide high levels of activity in the relevant matrices during the wine making process. These proteases ensure a more complete degradation of those proteins responsible for wine hazing than other considered proteases. The process is also fast, requiring only a short incubation time, making it a practical solution for winemakers. Additionally, these proteases are safe to use in consumable products as they degrade relatively rapidly into their component amino acids and/or into short peptides chains prior to bottling.

These advantages make the present disclosure a commercially desirable solution for preventing or reducing heat-induced protein haze formation or precipitate formation in wine; thereby solving a known problem in the wine industry.

### Proteases

The present disclosure provides methods of dehazing wine by treatment with one or more proteases. The compositions and methods described herein generally utilize an acid protease. In some embodiments, the acid protease is an S53 family protease. The term “S53 family proteases” and the like, generally refers to and includes the family of serine proteases found in prokaryotes and eukaryotes. In embodiments, the term “S53 family proteases” and the like refers to and includes proteases within and/or identified by MEROPS Accession MER0000995 (e.g., sedolisin, sedolisin-b, tripeptidyl-peptidase I, pro-Kumamolisin, Kumamolisin, Kumamolisin-B, physarolisin, aorsin, physarolisin II, Kumamolisin-As, grifolisin, scytalidolisin, among others). In various embodiments, the S53 family protease or acid protease is a pro-Kumamolisin protease. The terms “pro-Kumamolisin”, “pro-Kumamolisin protease”, and the like generally refers to and includes the thermostable calcium-dependent endopeptidase derived from an acid/thermophilic Bacillus (Bacillus novo sp. MN-32). In numerous embodiments, the term “pro-Kumamolisin” and the like refers to and includes NCBI Gene ID: 18765799 (NCBI Reference Sequence XP_007297753.1, XM_007297691.1 to XP_007297753, and/or NW_006763082.1 (137488 . . . 139728).

In several embodiments, a pro-Kumamolisin comprises or consists of an amino acid sequence as set forth in any one of SEQ ID NO: 1 to SEQ ID NO: 20. In many embodiments, a pro-Kumamolisin comprises or consists of an amino acid sequence having equal to or greater than 80% sequence identity to any one of SEQ ID NO: 1 to SEQ ID NO: 20. In some embodiments, a pro-Kumamolisin comprises or consists of an amino acid sequence having equal to or greater than 85% sequence identity to any one of SEQ ID NO: 1 to SEQ ID NO: 20. In some embodiments, a pro-Kumamolisin comprises or consists of an amino acid sequence having equal to or greater than 90% sequence identity to any one of SEQ ID NO: 1 to SEQ ID NO: 20. In embodiments, a pro-Kumamolisin comprises or consists of an amino acid sequence having equal to or greater than 95% sequence identity to any one of SEQ ID NO: 1 to SEQ ID NO: 20. In various embodiments, a pro-Kumamolisin comprises or consists of an amino acid sequence having equal to or greater than 97% sequence identity to any one of SEQ ID NO: 1 to SEQ ID NO: 20. In numerous embodiments, a pro-Kumamolisin comprises or consists of an amino acid sequence having equal to or greater than 98% sequence identity to any one of SEQ ID NO: 1 to SEQ ID NO: 20. In several embodiments, a pro-Kumamolisin comprises or consists of an amino acid sequence having equal to or greater than 99% sequence identity to any one of SEQ ID NO: 1 to SEQ ID NO: 20.

The determination of percent identity or percent similarity between two sequences can be accomplished using a mathematical algorithm. In many embodiments, a non-limiting example of a mathematical algorithm utilized for the comparison of two sequences is the algorithm of Karlin and Altschul, 1990, Proc. Natl. Acad. Sci. USA 87:2264-2268, modified as in Karlin and Altschul, 1993, Proc. Natl. Acad. Sci. USA 90:5873-5877. Such an algorithm is incorporated into the NBLAST and XBLAST programs of Altschul et al., 1990, J. Mol. Biol. 215:403-410. Alternatively, in some embodiments, PSI-Blast can be used to perform an iterated search which detects distant relationships between molecules (Id.). When utilizing BLAST, Gapped BLAST, and PSI-Blast programs, the default parameters of the respective programs (e.g., XBLAST and NBLAST) can be used. In some embodiments, a non-limiting example of a mathematical algorithm utilized for the comparison of sequences is the algorithm of Myers and Miller, CABIOS (1989). Such an algorithm is incorporated into the ALIGN program (version 2.0) which is part of the GCG sequence alignment software package. Additional algorithms for sequence analysis are known in the art and include ADVANCE and ADAM as described in Torellis and Robotti, 1994, Coinput. Appl. Biosci. 10:3-5; and FASTA described in Pearson and Lipman, 1988, Proc. Natl. Acad. Sci. USA 85:2444-8. In embodiments, sequence alignment is be carried out using the CLUSTAL algorithm, as described by Higgins et al., 1996, Methods Enzymol. 266:383-402.

In various embodiments, the pro-Kumamolisin protease has an active site comprising one, two, three, four, five, six, seven, eight, nine, ten, eleven, twelve, thirteen, fourteen, fifteen, sixteen, or seventeen residues at position 266, at position 295, at position 316, at position 317, at position 318, at position 349, at position 350, at position 351, at position 352, at position 353, at position 354, at position 367, at position 463, at position 464, at position 465, at position 466, and/or at position 467 each with respect to one of SEQ ID NO: 1 to SEQ ID NO: 20. In numerous embodiments, the pro-Kumamolisin protease comprises an active site wherein at position 266, the amino acid is E or D; at position 295, the amino acid is F, I, or L; at position 316, the amino acid is S; at position 317, the amino acid is W, L, T, or F; at position 318, the amino acid is G; at position 349, the amino acid is A or S; at position 350, the amino acid is A, S, T, or C; at position 351, the amino acid is G; at position 352, the amino acid is D; at position 353, the amino acid is S, N, A, R, E, H, or D; at position 354, the amino acid is G; at position 367, the amino acid is D, S, E, or N; at position 463, the amino acid is G; at position 464, the amino acid is G; at position 465, the amino acid is T; at position 466, the amino acid is S; at position 467, the amino acid is A, L, or W each with respect to one of SEQ ID NO: 1 to SEQ ID NO: 20. In several embodiments, the active site of a pro-Kumamolisin protease comprises amino acid residues E266 or D266; F295, 1295, or L295; S316; W317, L317, T317, or F317; G318; A349, S349; A350, S350, T350, or C350; G351; D352; S353, N353, A353, R353, E353, H353, or D353; G354; D367, S367, E367, or N367; G463; G464; T465; and S466; A467, L467, or W467 of any one of SEQ ID NO: 1 to SEQ ID NO: 20. In many embodiments, the pro-Kumamolisin protease comprises an active site comprising one or more amino acid substitutions, e.g., one, two, three, four, five, six, seven, eight, nine, ten, eleven, twelve, thirteen, fourteen, fifteen, sixteen, or seventeen substitutions, selected from E266, D266, F295, 1295, L295, S316, W317, L317, T317, F317, G318, A349, S349, A350, S35O, T350, C350, G351, D352, S353, N353, A353, R353, E353, H353, D353, G354, D367, S367, E367, N367, G463, G464, T465, S466, A467, L467, and W467 with respect to any one of SEQ ID NO: 1 to SEQ ID NO: 20. In some embodiments, the pro-Kumamolisin protease comprises or consists of an amino acid sequence having at least 85%, at least 90%, at least 95%, at least 98%, or at least 99% sequence identity to any one of SEQ ID NO: 1 to SEQ ID NO: 20 and comprises an active site having one or more amino acid substitutions, e.g., one, two, three, four, five, six, seven, eight, nine, ten, eleven, twelve, thirteen, fourteen, fifteen, sixteen, or seventeen substitutions of residues, selected from E266, D266, F295, 1295, L295, S316, W317, L317, T317, F317, G318, A349, S349, A350, S350, T350, C350, G351, D352, S353, N353, A353, R353, E353, H353, D353, G354, D367, S367, E367, N367, G463, G464, T465, S466, A467, L467, and W467 with respect to any one of SEQ ID NO: 1 to SEQ ID NO: 20. In some embodiments, the active site comprises between one and five amino acid substitutions.

In embodiments, the pro-Kumamolisin protease comprises or consists of an amino acid sequence having at least 85%, at least 90%, at least 95%, at least 98%, or at least 99% sequence identity to SEQ ID NO: 14 and comprises an active site having one or more amino acid substitutions, e.g., one, two, three, four, five, six, seven, eight, nine, ten, eleven, twelve, thirteen, fourteen, fifteen, sixteen, or seventeen substitutions of residues, selected from E266, D266, F295, 1295, L295, S316, W317, L317, T317, F317, G318, A349, S349, A35O, S350, T35O, C350, G351, D352, S353, N353, A353, R353, E353, H353, D353, G354, D367, S367, E367, N367, G463, G464, T465, S466, A467, L467, and W467 with respect to SEQ ID NO: 14.

In various embodiments, the pro-Kumamolisin protease comprises or consists of an amino acid sequence having at least 85%, at least 90%, at least 95%, at least 98%, or at least 99% sequence identity to SEQ ID NO: 8 and comprises an active site having one or more amino acid substitutions, e.g., one, two, three, four, five, six, seven, eight, nine, ten, eleven, twelve, thirteen, fourteen, fifteen, sixteen, or seventeen substitutions of residues, selected from E266, D266, F295, 1295, L295, S316, W317, L317, T317, F317, G318, A349, S349, A35O, S350, T35O, C350, G351, D352, S353, N353, A353, R353, E353, H353, D353, G354, D367, S367, E367, N367, G463, G464, T465, S466, A467, L467, and W467 with respect to SEQ ID NO: 8.

In numerous embodiments, the pro-Kumamolisin protease comprises or consists of an amino acid sequence having at least 85%, at least 90%, at least 95%, at least 98%, or at least 99% sequence identity to SEQ ID NO: 1 and comprises an active site having one or more amino acid substitutions, e.g., one, two, three, four, five, six, seven, eight, nine, ten, eleven, twelve, thirteen, fourteen, fifteen, sixteen, or seventeen substitutions of residues, selected from E266, D266, F295, 1295, L295, S316, W317, L317, T317, F317, G318, A349, S349, A35O, S350, T35O, C350, G351, D352, S353, N353, A353, R353, E353, H353, D353, G354, D367, S367, E367, N367, G463, G464, T465, S466, A467, L467, and W467 with respect to SEQ ID NO: 1.

In several embodiments, the active site comprises between two and five amino acid substitutions. In many embodiments, the active site comprises between three and five amino acid substitutions.

In some embodiments, the active site comprises one amino acid substitution. In some embodiments, the active site comprises two amino acid substitutions. In embodiments, the active site comprises three amino acid substitutions. In various embodiments, the active site comprises four amino acid substitutions. In numerous embodiments, the active site comprises five amino acid substitutions. In embodiments, the active site substitution does not reduce the stability, specificity, and/or activity of the protease. In embodiments, the active site substitution increases the stability, specificity, and/or activity of the protease.

An amino acid generally refers to and/or includes naturally occurring amino acids, unnatural amino acids, amino acid analogues and amino acid mimetics that function in a manner similar to a naturally occurring amino acids. Amino acids are generally referred to herein by either their name, the commonly known three letter symbols, or by the one-letter symbols recommended by the IUPAC-IUB Biochemical Nomenclature Commission. As used herein, naturally occurring amino acids include and/or refer to amino acids which are generally found in nature and are not manipulated by man. In several embodiments, naturally occurring includes and/or further refers to the 20 conventional amino acids; alanine (A or Ala), cysteine (C or Cys), aspartic acid (D or Asp), glutamic acid (E or Glu), phenylalanine (F or Phe), glycine (G or Gly), histidine (H or His), isoleucine (I or He), lysine (K or Lys), leucine (L or Leu), methionine (M or Met), asparagine (N or Asn), proline (P or Pro), glutamine (Q or Gin), arginine (R or Arg), serine (S or Ser), threonine (T or Thr), valine (V or Vai), tryptophan (W or Tip), and tyrosine (Y or Tyr).

In many embodiments, a non-polar amino acid can be substituted and replaced with another nonpolar amino acid, wherein non-polar amino acids include alanine, leucine, isoleucine, valine, glycine, proline, phenylalanine, tryptophan, and methionine. In some embodiments, a neutrally charged polar amino acids can be substituted and replaced with another neutrally charged polar amino acid, wherein neutrally charged polar amino acids include serine, threonine, cysteine, tyrosine, asparagine, and glutamine. In some embodiments, a positively charged amino acid can be substituted and replaced with another positively charged amino acid, wherein positively charged amino acids include arginine, lysine, and histidine. In embodiments, a negatively charged amino acid can be substituted and replaced with another negatively charged amino acid, wherein negatively charged amino acids include aspartic acid and glutamic acid. As used herein, a peptide includes and/or refers to any of various natural or synthetic compounds containing two or more amino acids joined by a peptide bond that link the carboxyl group of one amino acid to the amino group of another. As also used herein, amino acid refers to and/or includes naturally occurring amino acids, unnatural amino acids, amino acid analogues and amino acid mimetics that function in a manner similar to a naturally occurring amino acids. Amino acids are generally referred to herein by either their name, the commonly known three letter symbols, or by the one-letter symbols recommended by the IUPAC-IUB Biochemical Nomenclature Commission. In embodiments, an amino acid substitution does not reduce the stability, specificity, and/or activity of the protease. In embodiments, an amino acid substitution increases the stability, specificity, and/or activity of the protease.

In various embodiments, the pro-Kumamolisin protease comprises one or more truncations of any one of SEQ ID NO: 1 to SEQ ID NO: 20, wherein the one or more truncations comprises an N-terminal truncation, a C-terminal truncation, or both an N-terminal and C-terminal truncation. In embodiments, a truncation docs not reduce the stability, specificity, and/or activity of the protease. In embodiments, a truncation increases the stability, specificity, and/or activity of the protease.

The pro-Kumamolisin protease can be added to a liquid thereby forming a composition comprising the pro-Kumamolisin protease. In numerous embodiments, the pro-Kumamolisin protease is present at a concentration ranging from about 0.001 mg/L to about 100 mg/L or from about 0.001 mg/L to about 1000 mg/L, e.g., from about 0.001 mg/L to about 0.01 mg/L; from about 0.01 mg/L to about 0.1 mg/L; from about 0.1 mg/L to about 1 mg/L; from about 1 mg/L to about 10 mg/L; from about 10 mg/L to about 100 mg/L; or from about 100 mg/L to about 1,000 mg/L. In these compositions, the liquid in the composition may be any solvent that can be added to a fermentable juice, wine, or other beverage of interest, e.g., water, wine, and juice, or a combination thereof, as a concentrate. Alternately, the composition is a fermentable juice, wine, or other beverage of interest (as defined herein) which comprises the pro-Kumamolisin protease.

In several embodiments, the juice is grape juice, apple juice, orange juice, berry (e.g., blackberry, blueberry, blackcurrant, redcurrant, cranberry, elderberry, gooseberry, mulberry, seaberry, raspberry, and white-currant) juice, pineapple juice, pomegranate juice, plum juice, dandelion juice, rose hip juice, lychee juice, pear juice, or cherry juice.

In many embodiments, the beverage of interest comprises fermented honey.

In some embodiments, the beverage of interest comprises fermented grain, e.g., rice, barley, corn, rye, or wheat.

In some embodiments, the pro-Kumamolisin protease is present in a composition at a concentration ranging from about 0.001 mg/L to about 0.01 mg/L, or at any specific concentration therebetween or in any range of concentrations therebetween. As examples, the pro-Kumamolisin protease is present at a concentration of about 0.001 mg/L, about 0.002 mg/L, about 0.003 mg/L, about 0.004 mg/L, about 0.005 mg/L, about 0.006 mg/L, about 0.007 mg/L, about 0.008 mg/L, about 0.009 mg/L, about 0.01 mg/L. The pro-Kumamolisin protease may be is present at a concentration of at least about 0.001 mg/L, at least about 0.002 mg/L, at least about 0.003 mg/L, at least about 0.004 mg/L, at least about 0.005 mg/L, at least about 0.006 mg/L, at least about 0.007 mg/L, at least about 0.008 mg/L, at least about 0.009 mg/L, at least about 0.01 mg/L. The pro-Kumamolisin protease can be is present at a concentration of at most about 0.001 mg/L, at most about 0.002 mg/L, at most about 0.003 mg/L, at most about 0.004 mg/L, at most about 0.005 mg/L, at most about 0.006 mg/L, at most about 0.007 mg/L, at most about 0.008 mg/L, at most about 0.009 mg/L, at most about 0.01 mg/L. In these compositions, the liquid in the composition may be any solvent that can be added to a fermentable juice, wine, or other beverage of interest, e.g., water, wine, and juice, or a combination thereof, as a concentrate. Alternately, the composition is a fermentable juice, wine, or other beverage of interest (as defined herein) which comprises the pro-Kumamolisin protease.

In embodiments, the pro-Kumamolisin protease is present in a composition at a concentration ranging from about 0.01 mg/L to about 0.1 mg/L, or at any specific concentration therebetween or in any range of concentrations therebetween. As examples, the pro-Kumamolisin protease is present at a concentration of about 0.01 mg/L, about 0.02 mg/L, about 0.03 mg/L, about 0.04 mg/L, about 0.05 mg/L, about 0.06 mg/L, about 0.07 mg/L, about 0.08 mg/L, about 0.09 mg/L, about 0.1 mg/L. The pro-Kumamolisin protease may be is present at a concentration of at least about 0.01 mg/L, at least about 0.02 mg/L, at least about 0.03 mg/L, at least about 0.04 mg/L, at least about 0.05 mg/L, at least about 0.06 mg/L, at least about 0.07 mg/L, at least about 0.08 mg/L, at least about 0.09 mg/L, at least about 0.1 mg/L. The pro-Kumamolisin protease can be is present at a concentration of at most about 0.01 mg/L, at most about 0.02 mg/L, at most about 0.03 mg/L, at most about 0.04 mg/L, at most about 0.05 mg/L, at most about 0.06 mg/L, at most about 0.07 mg/L, at most about 0.08 mg/L, at most about 0.09 mg/L, at most about 0.1 mg/L. In these compositions, the liquid in the composition may be any solvent that can be added to a fermentable juice, wine, or other beverage of interest, e.g., water, wine, and juice, or a combination thereof, as a concentrate. Alternately, the composition is a fermentable juice, wine, or other beverage of interest (as defined herein) which comprises the pro-Kumamolisin protease.

In various embodiments, the pro-Kumamolisin protease is present in a composition at a concentration ranging from about 0.1 mg/L to about 1 mg/L, or at any specific concentration therebetween or in any range of concentrations therebetween. As examples, the pro-Kumamolisin protease is present at a concentration of about 0.1 mg/L, about 0.2 mg/L, about 0.3 mg/L, about 0.4 mg/L, about 0.5 mg/L, about 0.6 mg/L, about 0.7 mg/L, about 0.8 mg/L, about 0.9 mg/L, about 1 mg/L. The pro-Kumamolisin protease may be is present at a concentration of at least about 0.1 mg/L, at least about 0.2 mg/L, at least about 0.3 mg/L, at least about 0.4 mg/L, at least about 0.5 mg/L, at least about 0.6 mg/L, at least about 0.7 mg/L, at least about 0.8 mg/L, at least about 0.9 mg/L, at least about 1 mg/L. The pro-Kumamolisin protease can be is present at a concentration of at most about 0.1 mg/L, at most about 0.2 mg/L, at most about 0.3 mg/L, at most about 0.4 mg/L, at most about 0.5 mg/L, at most about 0.6 mg/L, at most about 0.7 mg/L, at most about 0.8 mg/L, at most about 0.9 mg/L, at most about 1 mg/L. In these compositions, the liquid in the composition may be any solvent that can be added to a fermentable juice, wine, or other beverage of interest, e.g., water, wine, and juice, or a combination thereof, as a concentrate. Alternately, the composition is a fermentable juice, wine, or other beverage of interest (as defined herein) which comprises the pro-Kumamolisin protease.

In numerous embodiments, the pro-Kumamolisin protease is present in a composition at a concentration ranging from about 1 mg/L to about 10 mg/L, or at any specific concentration therebetween or in any range of concentrations therebetween. As examples, the pro-Kumamolisin protease is present at a concentration of about 1 mg/L, about 2 mg/L, about 3 mg/L, about 4 mg/L, about 5 mg/L, about 6 mg/L, about 7 mg/L, about 8 mg/L, about 9 mg/L, about 10 mg/L. The pro-Kumamolisin protease may be is present at a concentration of at least about 1 mg/L, at least about 2 mg/L, at least about 3 mg/L, at least about 4 mg/L, at least about 5 mg/L, at least about 6 mg/L, at least about 7 mg/L, at least about 8 mg/L, at least about 9 mg/L, at least about 10 mg/L. The pro-Kumamolisin protease can be is present at a concentration of at most about 1 mg/L, at most about 2 mg/L, at most about 3 mg/L, at most about 4 mg/L, at most about 5 mg/L, at most about 6 mg/L, at most about 7 mg/L, at most about 8 mg/L, at most about 9 mg/L, at most about 10 mg/L. In these compositions, the liquid in the composition may be any solvent that can be added to a fermentable juice, wine, or other beverage of interest, e.g., water, wine, and juice, or a combination thereof, as a concentrate. Alternately, the composition is a fermentable juice, wine, or other beverage of interest (as defined herein) which comprises the pro-Kumamolisin protease.

In several embodiments, the pro-Kumamolisin protease is present in a composition at a concentration ranging from about 10 mg/L to about 100 mg/L, or at any specific concentration therebetween or in any range of concentrations therebetween. As examples, the pro-Kumamolisin protease is present at a concentration of about 10 mg/L, about 20 mg/L, about 30 mg/L, about 40 mg/L, about 50 mg/L, about 60 mg/L, about 70 mg/L, about 80 mg/L, about 90 mg/L, about 100 mg/L. The pro-Kumamolisin protease may be is present at a concentration of at least about 10 mg/L, at least about 20 mg/L, at least about 30 mg/L, at least about 40 mg/L, at least about 50 mg/L, at least about 60 mg/L, at least about 70 mg/L, at least about 80 mg/L, at least about 90 mg/L, at least about 100 mg/L. The pro-Kumamolisin protease can be is present at a concentration of at most about 10 mg/L, at most about 20 mg/L, at most about 30 mg/L, at most about 40 mg/L, at most about 50 mg/L, at most about 60 mg/L, at most about 70 mg/L, at most about 80 mg/L, at most about 90 mg/L, at most about 100 mg/L. In these compositions, the liquid in the composition may be any solvent that can be added to a fermentable juice, wine, or other beverage of interest, e.g., water, wine, and juice, or a combination thereof, as a concentrate. Alternately, the composition is a fermentable juice, wine, or other beverage of interest (as defined herein) which comprises the pro-Kumamolisin protease.

In many embodiments, the pro-Kumamolisin protease is present in a composition at a concentration ranging from about 100 mg/L to about 1,000 mg/L, or at any specific concentration therebetween or in any range of concentrations therebetween. As examples, the pro-Kumamolisin protease is present at a concentration of about 100 mg/L, about 200 mg/L, about 300 mg/L, about 400 mg/L, about 500 mg/L, about 600 mg/L, about 700 mg/L, about 800 mg/L, about 900 mg/L, about 1,000 mg/L. The pro-Kumamolisin protease may be is present at a concentration of at least about 100 mg/L, at least about 200 mg/L, at least about 300 mg/L, at least about 400 mg/L, at least about 500 mg/L, at least about 600 mg/L, at least about 700 mg/L, at least about 800 mg/L, at least about 900 mg/L, at least about 1,000 mg/L. The pro-Kumamolisin protease can be is present at a concentration of at most about 100 mg/L, at most about 200 mg/L, at most about 300 mg/L, at most about 400 mg/L, at most about 500 mg/L, at most about 600 mg/L, at most about 700 mg/L, at most about 800 mg/L, at most about 900 mg/L, at most about 1,000 mg/L. In these compositions, the liquid in the composition may be any solvent that can be added to a fermentable juice, wine, or other beverage of interest, e.g., water, wine, and juice, or a combination thereof, as a concentrate. Alternately, the composition is a fermentable juice, wine, or other beverage of interest (as defined herein) which comprises the pro-Kumamolisin protease.

In some embodiments, the pro-Kumamolisin protease is present in a composition at a concentration ranging from about 1,000 mg/L to about 10,000 mg/L, or at any specific concentration therebetween or in any range of concentrations therebetween. As examples, the pro-Kumamolisin protease is present at a concentration of about 1,000 mg/L, about 2,000 mg/L, about 3,000 mg/L, about 4,000 mg/L, about 5,000 mg/L, about 6,000 mg/L, about 7,000 mg/L, about 8,000 mg/L, about 9,000 mg/L, about 10,000 mg/L. The pro-Kumamolisin protease may be is present at a concentration of at least about 1,000 mg/L, at least about 2,000 mg/L, at least about 3,000 mg/L, at least about 4,000 mg/L, at least about 5,000 mg/L, at least about 6,000 mg/L, at least about 7,000 mg/L, at least about 8,000 mg/L, at least about 9,000 mg/L, at least about 10,000 mg/L. The pro-Kumamolisin protease can be is present at a concentration of at most about 1,000 mg/L, at most about 2,000 mg/L, at most about 3,000 mg/L, at most about 4,000 mg/L, at most about 5,000 mg/L, at most about 6,000 mg/L, at most about 7,000 mg/L, at most about 8,000 mg/L, at most about 9,000 mg/L, at most about 10,000 mg/L. In these compositions, the liquid in the composition may be any solvent that can be added to a fermentable juice, wine, or other beverage of interest, e.g., water, wine, and juice, or a combination thereof, as a concentrate. Alternately, the composition is a fermentable juice, wine, or other beverage of interest (as defined herein) which comprises the pro-Kumamolisin protease.

In some embodiments, the pro-Kumamolisin protease is present in a composition at a concentration ranging from about 10 mg/L to about 100 mg/L, or at any specific concentration therebetween or in any range of concentrations therebetween. As examples, the pro-Kumamolisin protease is present at a concentration of about 10 mg/L, about 20 mg/L, about 30 mg/L, about 40 mg/L, about 50 mg/L, about 60 mg/L, about 70 mg/L, about 80 mg/L, about 90 mg/L, about 100 mg/L. The pro-Kumamolisin protease may be is present at a concentration of at least about 10 mg/L, at least about 20 mg/L, at least about 30 mg/L, at least about 40 mg/L, at least about 50 mg/L, at least about 60 mg/L, at least about 70 mg/L, at least about 80 mg/L, at least about 90 mg/L, at least about 100 mg/L. The pro-Kumamolisin protease can be is present at a concentration of at most about 10 mg/L, at most about 20 mg/L, at most about 30 mg/L, at most about 40 mg/L, at most about 50 mg/L, at most about 60 mg/L, at most about 70 mg/L, at most about 80 mg/L, at most about 90 mg/L, at most about 100 mg/L. In these compositions, the liquid in the composition may be any solvent that can be added to a fermentable juice, wine, or other beverage of interest, e.g., water, wine, and juice, or a combination thereof, as a concentrate. Alternately, the composition is a fermentable juice, wine, or other beverage of interest (as defined herein) which comprises the pro-Kumamolisin protease.

Any composition or method disclosed herein is applicable to any herein-disclosed composition or method. In other words, any aspect or embodiment described herein can be combined with any other aspect or embodiment as disclosed herein.

### Methods

Any of the pro-Kumamolisin proteases described herein can be used in the methods provided. In embodiments, the pro-Kumamolisin protease comprises or consists of an amino acid sequence having at least 85% sequence identity to any one of SEQ ID NO: 1 to SEQ ID NO: 20. In various embodiments, the pro-Kumamolisin protease comprises or consists of an amino acid sequence having at least 95% sequence identity to any one of SEQ ID NO: 1 to SEQ ID NO: 20. In several embodiments, the pro-Kumamolisin protease comprises or consists of an amino acid sequence having at least 98% sequence identity to any one of SEQ ID NO: 1 to SEQ ID NO: 20.

In numerous embodiments, the pro-Kumamolisin protease comprises an active site wherein at position 266, the amino acid is E or D; at position 295, the amino acid is F, I, or L; at position 316, the amino acid is S; at position 317, the amino acid is W, L, T, or F; at position 318, the amino acid is G; at position 349, the amino acid is A or S; at position 350, the amino acid is A, S, T, or C; at position 351, the amino acid is G; at position 352, the amino acid is D; at position 353, the amino acid is S, N, A, R, E, H, or D; at position 354, the amino acid is G; at position 367, the amino acid is D, S, E, or N; at position 463, the amino acid is G; at position 464, the amino acid is G; at position 465, the amino acid is T; at position 466, the amino acid is S; at position 467, the amino acid is A, L, or W each with respect to one of SEQ ID NO: 1 to SEQ ID NO: 20. In many embodiments, the pro-Kumamolisin protease comprises or consists of an amino acid sequence of any one of SEQ ID NO: 1 to SEQ ID NO: 20. In some embodiments, the pro-Kumamolisin protease comprises an active site comprising amino acid residues E266 or D266; F295, 1295, or L295; S316; W317, L317, T317, or F317; G318; A349, S349; A35O, S350, T350, or C350; G351; D352; S353, N353, A353, R353, E353, H353, or D353; G354; D367, S367, E367, or N367; G463; G464; T465; and/or S466; A467, L467, or W467 with respect to any one of SEQ ID NO: 1 to SEQ ID NO: 20. In some embodiments, the pro-Kumamolisin protease comprises an active site comprising one or more amino acid substitutions, e.g., one, two, three, four, five, six, seven, eight, nine, ten, eleven, twelve, thirteen, fourteen, fifteen, sixteen, or seventeen substitutions, selected from E266, D266, F295, 1295, L295, S316, W317, L317, T317, F317, G318, A349, S349, A35O, S350, T350, C350, G351, D352, S353, N353, A353, R353, E353, H353, D353, G354, D367, S367, E367, N367, G463, G464, T465, S466, A467, L467, and W467 with respect to any one of SEQ ID NO: 1 to SEQ ID NO: 20. In embodiments, the pro-Kumamolisin protease comprises or consists of an amino acid sequence having at least 85%, at least 90%, at least 95%, at least 98%, or at least 99% sequence identity to any one of SEQ ID NO: 1 to SEQ ID NO: 20 and comprises an active site having one or more amino acid substitutions, e.g., one, two, three, four, five, six, seven, eight, nine, ten, eleven, twelve, thirteen, fourteen, fifteen, sixteen, or seventeen substitutions of residues, selected from E266, D266, F295, 1295, L295, S316, W317, L317, T317, F317, G318, A349, S349, A350, S350, T350, C350, G351, D352, S353, N353, A353, R353, E353, H353, D353, G354, D367, S367, E367, N367, G463, G464, T465, S466, A467, L467, and W467 with respect to any one of SEQ ID NO: 1 to SEQ ID NO: 20. In various embodiments, the active site comprises between one and five amino acid substitutions. In embodiments, the active site substitution does not reduce the stability, specificity, and/or activity of the protease. In embodiments, the active site substitution increases the stability, specificity, and/or activity of the protease. In several embodiments, the pro-Kumamolisin protease comprises one or more truncations of any one of SEQ ID NO: 1 to SEQ ID NO: 20, wherein the one or more truncations comprises an N-terminal truncation, a C-terminal truncation, or both an N-terminal and C-terminal truncation. In embodiments, the truncation does not reduce the stability, specificity, and/or activity of the protease. In embodiments, the truncation increases the stability, specificity, and/or activity of the protease.

In numerous embodiments, the pro-Kumamolisin protease comprises or consists of an amino acid sequence having at least 85%, at least 90%, at least 95%, at least 98%, or at least 99% sequence identity to SEQ ID NO: 14 and comprises an active site having one or more amino acid substitutions, e.g., one, two, three, four, five, six, seven, eight, nine, ten, eleven, twelve, thirteen, fourteen, fifteen, sixteen, or seventeen substitutions of residues, selected from E266, D266, F295, 1295, L295, S316, W317, L317, T317, F317, G318, A349, S349, A350, S35O, T350, C350, G351, D352, S353, N353, A353, R353, E353, H353, D353, G354, D367, S367, E367, N367, G463, G464, T465, S466, A467, L467, and W467 with respect to SEQ ID NO: 14. In many embodiments, the active site comprises between one and five amino acid substitutions. In embodiments, the active site substitution does not reduce the stability, specificity, and/or activity of the protease. In embodiments, the active site substitution increases the stability, specificity, and/or activity of the protease. In some embodiments, the pro-Kumamolisin protease comprises one or more truncations of SEQ ID NO: 14, wherein the one or more truncations comprises an N-terminal truncation, a C-terminal truncation, or both an N-terminal and C-terminal truncation. In embodiments, the truncation does not reduce the stability, specificity, and/or activity of the protease. In embodiments, the truncation increases the stability, specificity, and/or activity of the protease.

In some embodiments, the pro-Kumamolisin protease comprises or consists of an amino acid sequence having at least 85%, at least 90%, at least 95%, at least 98%, or at least 99% sequence identity to SEQ ID NO: 8 and comprises an active site having one or more amino acid substitutions, e.g., one, two, three, four, five, six, seven, eight, nine, ten, eleven, twelve, thirteen, fourteen, fifteen, sixteen, or seventeen substitutions of residues, selected from E266, D266, F295, 1295, L295, S316, W317, L317, T317, F317, G318, A349, S349, A35O, S350, T35O, C350, G351, D352, S353, N353, A353, R353, E353, H353, D353, G354, D367, S367, E367, N367, G463, G464, T465, S466, A467, L467, and W467 with respect to SEQ ID NO: 8. In embodiments, the active site comprises between one and five amino acid substitutions. In embodiments, the active site substitution does not reduce the stability, specificity, and/or activity of the protease. In embodiments, the active site substitution increases the stability, specificity, and/or activity of the protease. In various embodiments, the pro-Kumamolisin protease comprises one or more truncations of SEQ ID NO: 8, wherein the one or more truncations comprises an N-terminal truncation, a C-terminal truncation, or both an N-terminal and C-terminal truncation. In embodiments, the truncation does not reduce the stability, specificity, and/or activity of the protease. In embodiments, the truncation increases the stability, specificity, and/or activity of the protease.

In several embodiments, the pro-Kumamolisin protease comprises or consists of an amino acid sequence having at least 85%, at least 90%, at least 95%, at least 98%, or at least 99% sequence identity to SEQ ID NO: 1 and comprises an active site having one or more amino acid substitutions, e.g., one, two, three, four, five, six, seven, eight, nine, ten, eleven, twelve, thirteen, fourteen, fifteen, sixteen, or seventeen substitutions of residues, selected from E266, D266, F295, 1295, L295, S316, W317, L317, T317, F317, G318, A349, S349, A35O, S350, T35O, C350, G351, D352, S353, N353, A353, R353, E353, H353, D353, G354, D367, S367, E367, N367, G463, G464, T465, S466, A467, L467, and W467 with respect to SEQ ID NO: 1. In numerous embodiments, the active site comprises between one and five amino acid substitutions. In embodiments, the active site substitution does not reduce the stability, specificity, and/or activity of the protease. In embodiments, the active site substitution increases the stability, specificity, and/or activity of the protease. In many embodiments, the pro-Kumamolisin protease comprises one or more truncations of SEQ ID NO: 1, wherein the one or more truncations comprises an N-terminal truncation, a C-terminal truncation, or both an N-terminal and C-terminal truncation. In embodiments, the truncation does not reduce the stability, specificity, and/or activity of the protease. In embodiments, the truncation increases the stability, specificity, and/or activity of the protease.

In some embodiments, the amount of pro-Kumamolisin added to a liquid (e.g., a fermentable juice, wine, or other beverage of interest as defined herein, or any solvent that can be added to a fermentable juice, wine, or other beverage of interest, including water, wine, and juice, or a combination thereof) is sufficient to form a composition having a concentration of pro-Kumamolisin ranging from about 0.001 mg/L to about 100 mg/L or from about 0.001 mg/L to about 1000 mg/L, e.g., from about 0.001 mg/L to about 0.01 mg/L; from about 0.01 mg/L to about 0.1 mg/L; from about 0.1 mg/L to about 1 mg/L; from about 1 mg/L to about 10 mg/L; from about 10 mg/L to about 100 mg/L; or from about 100 mg/L to about 1,000 mg/L. In these compositions, the liquid in the composition may be any solvent that can be added to a fermentable juice, wine, or other beverage of interest, e.g., water, wine, and juice, or a combination thereof, as a concentrate. Alternately, the composition is a fermentable juice, wine, or other beverage of interest (as defined herein) which comprises the pro-Kumamolisin protease.

In some embodiments, the juice is grape juice, apple juice, orange juice, berry (e.g., blackberry, blueberry, blackcurrant, redcurrant, cranberry, elderberry, gooseberry, mulberry, seaberry, raspberry, and white-currant) juice, pineapple juice, pomegranate juice, plum juice, dandelion juice, rose hip juice, lychee juice, pear juice, or cherry juice.

In embodiments, the beverage of interest comprises fermented honey.

In various embodiments, the beverage of interest comprises fermented grain, e.g., rice, barley, corn, rye, or wheat.

The amount of pro-Kumamolisin protease added to a liquid is sufficient to form a composition having a concentration of pro-Kumamolisin ranging from about 0.001 mg/L to about 0.01 mg/L, or at any specific concentration therebetween or in any range of concentrations therebetween. As examples, the pro-Kumamolisin protease is present at a concentration of about 0.001 mg/L, about 0.002 mg/L, about 0.003 mg/L, about 0.004 mg/L, about 0.005 mg/L, about 0.006 mg/L, about 0.007 mg/L, about 0.008 mg/L, about 0.009 mg/L, about 0.01 mg/L. The pro-Kumamolisin protease may be is present at a concentration of at least about 0.001 mg/L, at least about 0.002 mg/L, at least about 0.003 mg/L, at least about 0.004 mg/L, at least about 0.005 mg/L, at least about 0.006 mg/L, at least about 0.007 mg/L, at least about 0.008 mg/L, at least about 0.009 mg/L, at least about 0.01 mg/L. The pro-Kumamolisin protease can be is present at a concentration of at most about 0.001 mg/L, at most about 0.002 mg/L, at most about 0.003 mg/L, at most about 0.004 mg/L, at most about 0.005 mg/L, at most about 0.006 mg/L, at most about 0.007 mg/L, at most about 0.008 mg/L, at most about 0.009 mg/L, at most about 0.01 mg/L. In these compositions, the liquid in the composition may be any solvent that can be added to a fermentable juice, wine, or other beverage of interest, e.g., water, wine, and juice, or a combination thereof, as a concentrate. Alternately, the composition is a fermentable juice, wine, or other beverage of interest (as defined herein) which comprises the pro-Kumamolisin protease. The amount of pro-Kumamolisin protease added can readily be calculated by a skilled artisan based on the desired concentration for a given volume of composition.

The amount of pro-Kumamolisin protease added to a liquid is sufficient to form a composition having a concentration of pro-Kumamolisin ranging from about 0.01 mg/L to about 0.1 mg/L, or at any specific concentration therebetween or in any range of concentrations therebetween. As examples, the pro-Kumamolisin protease is present at a concentration of about 0.01 mg/L, about 0.02 mg/L, about 0.03 mg/L, about 0.04 mg/L, about 0.05 mg/L, about 0.06 mg/L, about 0.07 mg/L, about 0.08 mg/L, about 0.09 mg/L, about 0.1 mg/L. The pro-Kumamolisin protease may be is present at a concentration of at least about 0.01 mg/L, at least about 0.02 mg/L, at least about 0.03 mg/L, at least about 0.04 mg/L, at least about 0.05 mg/L, at least about 0.06 mg/L, at least about 0.07 mg/L, at least about 0.08 mg/L, at least about 0.09 mg/L, at least about 0.1 mg/L. The pro-Kumamolisin protease can be is present at a concentration of at most about 0.01 mg/L, at most about 0.02 mg/L, at most about 0.03 mg/L, at most about 0.04 mg/L, at most about 0.05 mg/L, at most about 0.06 mg/L, at most about 0.07 mg/L, at most about 0.08 mg/L, at most about 0.09 mg/L, at most about 0.1 mg/L. In these compositions, the liquid in the composition may be any solvent that can be added to a fermentable juice, wine, or other beverage of interest, e.g., water, wine, and juice, or a combination thereof, as a concentrate. Alternately, the composition is a fermentable juice, wine, or other beverage of interest (as defined herein) which comprises the pro-Kumamolisin protease. The amount of pro-Kumamolisin protease added can readily be calculated by a skilled artisan based on the desired concentration for a given volume of composition.

The amount of pro-Kumamolisin protease added to a liquid is sufficient to form a composition having a concentration of pro-Kumamolisin ranging from about 0.1 mg/L to about 1 mg/L, or at any specific concentration therebetween or in any range of concentrations therebetween. As examples, the pro-Kumamolisin protease is present at a concentration of about 0.1 mg/L, about 0.2 mg/L, about 0.3 mg/L, about 0.4 mg/L, about 0.5 mg/L, about 0.6 mg/L, about 0.7 mg/L, about 0.8 mg/L, about 0.9 mg/L, about 1 mg/L. The pro-Kumamolisin protease may be is present at a concentration of at least about 0.1 mg/L, at least about 0.2 mg/L, at least about 0.3 mg/L, at least about 0.4 mg/L, at least about 0.5 mg/L, at least about 0.6 mg/L, at least about 0.7 mg/L, at least about 0.8 mg/L, at least about 0.9 mg/L, at least about 1 mg/L. The pro-Kumamolisin protease can be is present at a concentration of at most about 0.1 mg/L, at most about 0.2 mg/L, at most about 0.3 mg/L, at most about 0.4 mg/L, at most about 0.5 mg/L, at most about 0.6 mg/L, at most about 0.7 mg/L, at most about 0.8 mg/L, at most about 0.9 mg/L, at most about 1 mg/L. In these compositions, the liquid in the composition may be any solvent that can be added to a fermentable juice, wine, or other beverage of interest, e.g., water, wine, and juice, or a combination thereof, as a concentrate. Alternately, the composition is a fermentable juice, wine, or other beverage of interest (as defined herein) which comprises the pro-Kumamolisin protease. The amount of pro-Kumamolisin protease added can readily be calculated by a skilled artisan based on the desired concentration for a given volume of composition.

The amount of pro-Kumamolisin protease added to a liquid is sufficient to form a composition having a concentration of pro-Kumamolisin ranging from about 1 mg/L to about 10 mg/L, or at any specific concentration therebetween or in any range of concentrations therebetween. As examples, the pro-Kumamolisin protease is present at a concentration of about 1 mg/L, about 2 mg/L, about 3 mg/L, about 4 mg/L, about 5 mg/L, about 6 mg/L, about 7 mg/L, about 8 mg/L, about 9 mg/L, about 10 mg/L. The pro-Kumamolisin protease may be is present at a concentration of at least about 1 mg/L, at least about 2 mg/L, at least about 3 mg/L, at least about 4 mg/L, at least about 5 mg/L, at least about 6 mg/L, at least about 7 mg/L, at least about 8 mg/L, at least about 9 mg/L, at least about 10 mg/L. The pro-Kumamolisin protease can be is present at a concentration of at most about 1 mg/L, at most about 2 mg/L, at most about 3 mg/L, at most about 4 mg/L, at most about 5 mg/L, at most about 6 mg/L, at most about 7 mg/L, at most about 8 mg/L, at most about 9 mg/L, at most about 10 mg/L. In these compositions, the liquid in the composition may be any solvent that can be added to a fermentable juice, wine, or other beverage of interest, e.g., water, wine, and juice, or a combination thereof, as a concentrate. Alternately, the composition is a fermentable juice, wine, or other beverage of interest (as defined herein) which comprises the pro-Kumamolisin protease. The amount of pro-Kumamolisin protease added can readily be calculated by a skilled artisan based on the desired concentration for a given volume of composition.

The amount of pro-Kumamolisin protease added to a liquid is sufficient to form a composition having a concentration of pro-Kumamolisin ranging from about 10 mg/L to about 100 mg/L, or at any specific concentration therebetween or in any range of concentrations therebetween. As examples, the pro-Kumamolisin protease is present at a concentration of about 10 mg/L, about 20 mg/L, about 30 mg/L, about 40 mg/L, about 50 mg/L, about 60 mg/L, about 70 mg/L, about 80 mg/L, about 90 mg/L, about 100 mg/L. The pro-Kumamolisin protease may be is present at a concentration of at least about 10 mg/L, at least about 20 mg/L, at least about 30 mg/L, at least about 40 mg/L, at least about 50 mg/L, at least about 60 mg/L, at least about 70 mg/L, at least about 80 mg/L, at least about 90 mg/L, at least about 100 mg/L. The pro-Kumamolisin protease can be is present at a concentration of at most about 10 mg/L, at most about 20 mg/L, at most about 30 mg/L, at most about 40 mg/L, at most about 50 mg/L, at most about 60 mg/L, at most about 70 mg/L, at most about 80 mg/L, at most about 90 mg/L, at most about 100 mg/L. In these compositions, the liquid in the composition may be any solvent that can be added to a fermentable juice, wine, or other beverage of interest, e.g., water, wine, and juice, or a combination thereof, as a concentrate. Alternately, the composition is a fermentable juice, wine, or other beverage of interest (as defined herein) which comprises the pro-Kumamolisin protease. The amount of pro-Kumamolisin protease added can readily be calculated by a skilled artisan based on the desired concentration for a given volume of composition.

The amount of pro-Kumamolisin protease added to a liquid is sufficient to form a composition having a concentration of pro-Kumamolisin ranging from about 100 mg/L to about 1,000 mg/L, or at any specific concentration therebetween or in any range of concentrations therebetween. As examples, the pro-Kumamolisin protease is present at a concentration of about 100 mg/L, about 200 mg/L, about 300 mg/L, about 400 mg/L, about 500 mg/L, about 600 mg/L, about 700 mg/L, about 800 mg/L, about 900 mg/L, about 1,000 mg/L. The pro-Kumamolisin protease may be is present at a concentration of at least about 100 mg/L, at least about 200 mg/L, at least about 300 mg/L, at least about 400 mg/L, at least about 500 mg/L, at least about 600 mg/L, at least about 700 mg/L, at least about 800 mg/L, at least about 900 mg/L, at least about 1,000 mg/L. The pro-Kumamolisin protease can be is present at a concentration of at most about 100 mg/L, at most about 200 mg/L, at most about 300 mg/L, at most about 400 mg/L, at most about 500 mg/L, at most about 600 mg/L, at most about 700 mg/L, at most about 800 mg/L, at most about 900 mg/L, at most about 1,000 mg/L. In these compositions, the liquid in the composition may be any solvent that can be added to a fermentable juice, wine, or other beverage of interest, e.g., water, wine, and juice, or a combination thereof, as a concentrate. Alternately, the composition is a fermentable juice, wine, or other beverage of interest (as defined herein) which comprises the pro-Kumamolisin protease. The amount of pro-Kumamolisin protease added can readily be calculated by a skilled artisan based on the desired concentration for a given volume of composition.

The amount of pro-Kumamolisin protease added to a liquid is sufficient to form a composition having a concentration of pro-Kumamolisin ranging from about 1,000 mg/L to about 10,000 mg/L, or at any specific concentration therebetween or in any range of concentrations therebetween. As examples, the pro-Kumamolisin protease is present at a concentration of about 1,000 mg/L, about 2,000 mg/L, about 3,000 mg/L, about 4,000 mg/L, about 5,000 mg/L, about 6,000 mg/L, about 7,000 mg/L, about 8,000 mg/L, about 9,000 mg/L, about 10,000 mg/L. The pro-Kumamolisin protease may be is present at a concentration of at least about 1,000 mg/L, at least about 2,000 mg/L, at least about 3,000 mg/L, at least about 4,000 mg/L, at least about 5,000 mg/L, at least about 6,000 mg/L, at least about 7,000 mg/L, at least about 8,000 mg/L, at least about 9,000 mg/L, at least about 10,000 mg/L. The pro-Kumamolisin protease can be is present at a concentration of at most about 1,000 mg/L, at most about 2,000 mg/L, at most about 3,000 mg/L, at most about 4,000 mg/L, at most about 5,000 mg/L, at most about 6,000 mg/L, at most about 7,000 mg/L, at most about 8,000 mg/L, at most about 9,000 mg/L, at most about 10,000 mg/L. In these compositions, the liquid in the composition may be any solvent that can be added to a fermentable juice, wine, or other beverage of interest, e.g., water, wine, and juice, or a combination thereof, as a concentrate. Alternately, the composition is a fermentable juice, wine, or other beverage of interest (as defined herein) which comprises the pro-Kumamolisin protease. The amount of pro-Kumamolisin protease added can readily be calculated by a skilled artisan based on the desired concentration for a given volume of composition.

The amount of pro-Kumamolisin protease added to a liquid is sufficient to form a composition having a concentration of pro-Kumamolisin ranging from about 10 mg/L to about 100 mg/L, or at any specific concentration therebetween or in any range of concentrations therebetween. As examples, the pro-Kumamolisin protease is present at a concentration of about 10 mg/L, about 20 mg/L, about 30 mg/L, about 40 mg/L, about 50 mg/L, about 60 mg/L, about 70 mg/L, about 80 mg/L, about 90 mg/L, about 100 mg/L. The pro-Kumamolisin protease may be is present at a concentration of at least about 10 mg/L, at least about 20 mg/L, at least about 30 mg/L, at least about 40 mg/L, at least about 50 mg/L, at least about 60 mg/L, at least about 70 mg/L, at least about 80 mg/L, at least about 90 mg/L, at least about 100 mg/L. The pro-Kumamolisin protease can be is present at a concentration of at most about 10 mg/L, at most about 20 mg/L, at most about 30 mg/L, at most about 40 mg/L, at most about 50 mg/L, at most about 60 mg/L, at most about 70 mg/L, at most about 80 mg/L, at most about 90 mg/L, at most about 100 mg/L. In these compositions, the liquid in the composition may be any solvent that can be added to a fermentable juice, wine, or other beverage of interest, e.g., water, wine, and juice, or a combination thereof, as a concentrate. Alternately, the composition is a fermentable juice, wine, or other beverage of interest (as defined herein) which comprises the pro-Kumamolisin protease. The amount of pro-Kumamolisin protease added can readily be calculated by a skilled artisan based on the desired concentration for a given volume of composition.

In several embodiments, the contacting or the adding of the protease to the beverage of interest results in removal of at least about 85% of heat-unstable proteins in the beverage.

In numerous embodiments, the contacting or the adding of the protease to the beverage of interest affects the beverage's taste, aroma, color, mouthfeel, and/or perception less than a dehazing method comprising contacting or the adding of bentonite.

In many embodiments, the contacting or the adding of the protease to the beverage of interest does not substantially affect the beverage's taste, aroma, color, mouthfeel, and/or perception.

In some embodiments, the methods comprise incubating the composition for 30 minutes to 72 hours, and any period time therebetween.

In some embodiments, the methods comprise incubating the composition for 3 days to 3 weeks, and any period time therebetween.

In embodiments, the methods comprise incubating the composition for 3 weeks to 3 months, and any period time therebetween.

In various embodiments, the methods comprise incubating the composition for 3 months to 3 years, and any period time therebetween.

In several embodiments, the methods comprise filtering the composition after treatment with the protease to remove residual haze-related precipitates.

In numerous embodiments, the methods comprise a step of adding a fining agent to the composition, thereby increasing clarity of the composition. In some cases, the fining agent is bentonite. The amount of fining agent used is less than needed to dehaze the beverage of interest in the absence of the protease and/or the amount of protease used is less than needed to dehaze the beverage of interest in the absence of the fining agent. The protease may be added to or contacted with the beverage of interest before adding the fining agent, the protease may be added to or contacted with the beverage of interest after adding the fining agent, or the protease may be added to or contacted with the beverage of interest simultaneous with the adding of the fining agent.

In many embodiments, the protease is immobilized on a solid support prior to contacting with the beverage of interest, e.g., wine. In some cases, the solid support is a column or another device that permits the beverage to flow through and to contact the immobilize protease.

In some embodiments, the methods further comprise monitoring protein content and/or optical density of a sample of the beverage of interest, e.g., wine, and/or the composition to assess the degree of haze reduction.

Any composition or method disclosed herein is applicable to any herein-disclosed composition or method. In other words, any aspect or embodiment described herein can be combined with any other aspect or embodiment as disclosed herein.

### Definitions

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

As used herein, the singular forms “a”, “an”, and “the” are intended to include the plural forms as well, unless the context clearly indicates otherwise. Furthermore, to the extent that the terms “including”, “includes”, “having”, “has”, “with”, or variants thereof are used in either the detailed description and/or the claims, such terms are intended to be inclusive in a manner similar to the term “comprising.”

The term “about” or “approximately” means within an acceptable error range for the particular value as determined by one of ordinary skill in the art, which will depend in pail on how the value is measured or determined, e.g., the limitations of the measurement system. For example, “about” can mean within 1 or more than 1 standard deviation, per the practice in the art. Alternatively, “about” can mean a range of up to 20%, up to 15%, up to 10%, up to 5%, or up to 1% of a given value. In some cases, the term “about” refers to +10% of a stated number or value. Alternatively, particularly with respect to biological systems or processes, the term can mean within an order of magnitude, preferably within 5-fold, and more preferably within 2-fold, of a value. Where particular values are described in the application and claims, unless otherwise stated the term “about” meaning within an acceptable error range for the particular value should be assumed.

As used herein, the phrases “at least one”, “one or more”, and “and/or” are open-ended expressions that are both conjunctive and disjunctive in operation. For example, each of the expressions “at least one of A, B and C”, “at least one of A, B, or C”, “one or more of A, B, and C”, “one or more of A, B, or C” and “A, B, and/or C” means A alone, B alone, C alone, A and B together, A and C together, B and C together, or A, B and C together.

As used herein, “or” may refer to “and”, “or,” or “and/or” and may be used both exclusively and inclusively. For example, the term “A or B” may refer to “A or B”, “A but not B”, “B but not A”, and “A and B”. In some cases, context may dictate a particular' meaning.

The terms “increased”, “increasing”, “increase”, “improved”, “improvement”, “improving” and the like, are used herein to generally means an increase by a statically significant amount. In some aspects, the terms “increased” or “improved” means an increase or improvement of at least 10% as compared to a reference level, for example an increase or improvement of at least about 10%, at least about 20%, or at least about 30%, or at least about 40%, or at least about 50%, or at least about 60%, or at least about 70%, or at least about 80%, or at least about 90% or up to and including a 100% increase or any improvement between 10-100% as compared to a reference level, standard, or control. Other examples of “increase” or “improvement” includes an increase of at least 2-fold, at least 5-fold, at least 10-fold, at least 20-fold, at least 50-fold, at least 100-fold, at least 1000-fold or more as compared to a reference level.

The terms “decreased”, “decreasing”, “decrease”, “reduced”, “reducing”, “reduce” and the like, are used herein generally to mean a decrease or reduction by a statistically significant amount. In some aspects, “decreased” or “reduced” means a reduction by at least 10% as compared to a reference level, for example a decrease or reduction by at least about 20%, or at least about 30%, or at least about 40%, or at least about 50%, or at least about 60%, or at least about 70%, or at least about 80%, or at least about 90% or up to and including a 100% decrease or reduction (e.g., absent level or non-detectable level as compared to a reference level), or any decrease or reduction between 10-100% as compared to a reference level.

Throughout this application, various embodiments may be presented in a range format. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

The section headings used herein are for organizational purposes only and are not to be construed as limiting the subject matter described.

As used herein, the words “comprising” (and any form of comprising, such as “comprise” and “comprises”), “having” (and any form of having, such as “have” and “has”), “including” (and any form of including, such as “include” and “includes”) or “containing” (and any form of containing, such as “contain” and “contains”), are inclusive or open-ended and do not exclude additional, unrecited elements or process steps. As also used herein, in any instance or embodiment described herein, “comprising” may be replaced with “consisting essentially of’ and/or “consisting of’, used herein, in any instance or embodiment described.

Any composition or method disclosed herein is applicable to any herein-disclosed composition or method. In other words, any aspect or embodiment described herein can be combined with any other aspect or embodiment as disclosed herein.

| SEQ ID NO | SEQUENCE | DESCRIPTION |
|---|---|---|
| 1 | MDETNFTSTNGSPQYIPVTGSARAIVPGATHAGHTD | Protein Peptidase |
| | DNEVLSVTLQLRRPSADELTAHVEALGTTPPANRKH | S53 precursor |
| | MTHDEFEASHGASDDDLNLVTAFATEQGLSVERINK | [Alicyclobacillus |
| | AAATVHVSGTAGAFNKAFHVQLGNYQHPDFTYRGY | ferrooxydans] |
| | DGPVHIPAHLTDIVTGVLGLDNRPQAKPHFRVYQEA | |
| | AVRSNALAAPISYTPTQVAALYNFPTNVDCSGQCIGII | |
| | ELGGGYSKSNLDQYFASLGVPTPTITSVSVDGGQNQP | |
| | TGSPNGPDGEVDLDIEVAASVAPGAHIAVYFAPNTD | |
| | AGFLDAITTAVHDKTNKPSVISISWGGPEMSWTTQA | |
| | MQAMNNAMQSAAALGVTITVAAGDNGSTDGVNDG | |
| | SFHVDFPASAPYALACGGTHLVGSGSTIESETVWND | |
| | GANGGATGGGVSSVFPVPSWQQKANVPPSANPGAG | |
| | TGRGVPDVSGDADPATGYQVLVDGQQFPIGGTSAV | |
| | APLWAGLVALANQTLGKPVGYINPLLYSIPAQDNAF | |
| | HDITQGNNDPNQTGQVYPAGPGWDACTGLGSPNGT | |
| | LLIQALGQIG | |
| 2 | MSKHPLMGSERAPFDGAQSVGKADPAERLEVTVLV | Protein Peptidase |
| | RRGSSDALRTRVSKLVAGNASDGHIQREDFAQQFGA | S53 precursor |
| | APNDMSAVRNFASQHGLSVVEEHAARRTVILSGTVA | [Caballeronia |
| | QFNDAFDVDLQQFEHAGGSYRGRTGPVHLPDELSGV | humi] |
| | VDAVLGLDNRPQARPHFRSRPPQGNVHWQSSRTGTT | |
| | SSTPLQLASLYDFPAGTGQGQCIAIIELGGGYRPADL | |
| | KAYFSKLGIASPKVTTVSVDHGKNHPTGDANGPDGE | |
| | VMLDIEIAGAIAPGAHIAVYFAPNTDAGFLDAVTTAI | |
| | HDTIRKPSVISISWGGPESAWTEQAMTAFDQAFQAA | |
| | AALGITVCVASGDNGSGDGVNDGADHVDFPASSPY | |
| | ALACGGTSVQAGKGAIAKETVWNDGANGGASGGG | |
| | VSSFFALPAWQEGLQAARAKGGTGALQMRGVPDVA | |
| | GNADPATGYDVRVDGSDMVIGGTSAVAPLWAGLV | |
| | ARINAGKNSPAGYLNPKLYKTAAGLTDITQGNNGDF | |
| | VASAGWDACTGLGRPDGNKLAGTFG | |
| 3 | MPQSQNRVVVRGSERQPMPKAHSQHALPPTERLEVT | Protein Peptidase |
| | VRLRPKAALASAAASSHAMADVPPSQRTYLSREELA | S53 precursor |
| | AQCGASEDDAQAVADFAHAHGLVVIHTDLARRSVL | [Hymenobacter |
| | LAGTAADFGAAFGTQLHQYSSPEGTYRGRTGTVTVP | psoromatis] |
| | APLADIVQGVFGLDDRRQAEPHFQVRPGPTPAPGAIV | |
| | ARAAGQSFTPPQLAQLYDFPGGLDGTGQTIAVIELGG | |
| | GFKPADLKAYFTGLNLPVPTVKVVSVNGGRNQPTN | |
| | ANSADGEVLLDIEVAAAVAPRAHLVVYFAPNTSQGF | |
| | LNAITTAVHDKVNNPGIISISWGGPESTWTGQAMDQ | |
| | FDQAFQEAAMLGVTVCVAAGDNGSADGVADGQPH | |
| | ADFPASSPFALACGGTKLTASGPTISSEVVWNEGPNS | |
| | ATGGGLSAHFPVPAYQQQLKFPTPPAGAKAGRGLPD | |
| | VAGDADPNTGYQVRVDGQNLVIGGTSAVAPLWAGL | |
| | LALLNQKLPKPVGFLNPLLYGSLAGQGVTRDITSGN | |
| | NGAFAAGPGWDACTGWGSPVGGKLLAALQGGAAV | |
| | A | |
| 4 | MESIMPSQPSSIPVRGSERAALPTAHVVGPAASDERL | Protein Peptidase |
| | EVTLRVRPRAQLHASASEAQSLRPPGERSYLSREQLA | S53 precursor |
| | SAHGAAPEDIAKVEAFAQSHGLQVVLTSAARRCVIV | [Caballeronia |
| | SGTVAALESAFAVKLQQYRFDGGSYRGRVGPVFVSP | jiangsuensis] |
| | EIGDIVEGVFGLDDRPQAIAHFKRSAHAVRAEDGAA | |
| | PHAGGASFTPPQLAKLYNYPGDTDGTGQCIGIVEFGG | |
| | AIRAADIRAYFKELGLPAPHVNTVLVDHAHMRSDDA | |
| | DAEVMLDIEVAAAIAPKAQIVVYFAPNTSQGFIDAFT | |
| | HAIHDTVHKPSVISVSWGGPEKDWSAQIKTQLDQVF | |
| | QDAAALGVTICAASGDAGSSDENPDALASIGLTPDG | |
| | LSHADFPASSPFALACGGTKLVASASAITSETVWNED | |
| | PVRSATGGGISDFFDVPGYQATANIPVSANPGGRKGR | |
| | GVPDIAADADPATGYLVRVHGQDAVIGGTSAVAPL | |
| | MAGLVALLNHKLGHPVGFLNPLLYRTAGITRDITQG | |
| | NNGAYAAGKGWDACTGLGVPDGAKLLDALM | |
| 5 | MSTRAARTTPSALADLRNEPRSPLPGSEKAALADTPA | Protein |
| | TTAAGIKPLRATAVAKAKPASSRKKITVSVVVPRTKP | Kumamolisin |
| | VTQAAVAGKHLTRAQFKSSHAAAPASVKAVOKFAK | precursor |
| | AFNLVSKAEPARSTVHLTGTVKDMQDAFGVTLQEH | [Terriglobus |
| | TVGAKTLRIRQGAIYLPDSVLPHVQAVLGLDNRPQA | roseus] |
| | KPHYRVGKARAAASTSFTPPQLAQLYGFPTSAKATG | |
| | QTIALIELGGGFRQADITAYFKSLGIAAPSVKAVLVD | |
| | GGKNAPSNANGADGEVMLDIEVAAAVAPGANIAVY | |
| | FAPNTDQGFVDAIATAAHDTTNKPTIISISWGGPESS | |
| | WTSQALTALDNACKDAAALGITVTAAAGDDGSDDG | |
| | VGDGKKHVDFPASSPNVLACGGTKLVASNGAITSEV | |
| | VWNETANKEGATGGGISTAFPQPTWQKSIAATKSGR | |
| | GVPDVAGDADPTTGYQVRVDGQNMVIGGTSAVAPL | |
| | WAGLIALSNATNKNAAGLPQAKLYSTTGQKAFRDIT | |
| | SGNNGAFKAAKGWDPCTGLGSPKAASIITLLATKSS | |
| | AKKKTSRAKA | |
| 6 | MAGVNEPYNAREDGIPLKSSARAVVPGVKLHGPTD | Protein Peptidase |
| | GASRLEITVVLRRRTELPSAAADGHLTAAELASEYG | S53 precursor |
| | ASDDDVRLATEVFTRLGADVVESDPASRRLRLSGTV | [Arthrobacter |
| | EQLSSIFGTTLEDATSTAPDGATVHYRHRLGELRIPAE | woluwensis] |
| | LNGIVIAVLGLDDRPQARAHFRMLPRTTAGTSYSPVE | |
| | LGRVYGFPDGTDGSGQTVAIIELGGGYAQADLDAYF | |
| | AGLGLATPQITSIGVDGGANQGGNDPQGADGEVLLD | |
| | IEVVGALAPKAAIQVYFAPNTDAGFLDAVVAATKAA | |
| | PCAISISWGQSEDQWTAQARDAFDQALADAAALGIT | |
| | TTVAAGDRGSSDGAADGKAHVDFPASSPHALACGG | |
| | TRLEADPATGAIRSETVWNEGPDSATGGGYSKVFPR | |
| | PSWQSPSAGKSGRGVPDVSAVADPQTGYRIRVDGKD | |
| | MVIGGTSAVAPLWAALIARFAQAGNRRFGLIQPSLY | |
| | AVSSGFRDVTVGDNGSYHAGPGWDACTGLGTPDGA | |
| | ALLAALKG | |
| 7 | MSTTAARRHHWPLANEARYLGPHEGTEPMDVTIVM | Protein Peptidase |
| | RHRGGRHPEPATWPHQPALPRADFGTQWGADPADA | S53 precursor |
| | DVIRKFASEHGLTEISCALNRRVMHLRGTPQQLQQA | [Dyella japonica] |
| | FGVAFGSYQMPDGGPAMIGCHLPPSLPSTISPSVIAVL | |
| | GLDKRPIARPHFRRAATPPATTYTPVQLGSLYQFPAN | |
| | LDGTGQTVAVIELGGGFTQTDFTSYMQSLGLKVPAV | |
| | NVVSVQGGTSQPGGDADGEVMLDVEVIGALAPGAA | |
| | IALYFAPNTDQGFYEAISQAAHDAANHPSVISISWGG | |
| | PEDNWSASSLSAMQSALQDAAAMGVTVTVACGDSG | |
| | YTDGESDNNPHVDFPASSPYALACGGTTLHGAGTAI | |
| | TSEVVWNDLSTNEGATGGGVSASFALPTWQQSSKVP | |
| | AGANGYVGRGLPDVAGDADPNTGYQVLVDGQSQVI | |
| | GGTSAVAPLWAALIARFNQQLGQSVGDPHAALYGIG | |
| | ESAFNDITQGNNGKFNATAGWDACTGLGTPKGQTL | |
| | LDALAALKGGGGTTAPSPPTNPTSPSKPTKPTKPKKP | |
| | TKPTKPSKPKPSK | |
| 8 | MANRKMFPNSVIAIPTSGVTAHGLIVSAADPQSRDEK | Protein Peptidase |
| | MDVSFSLGIPPALEKELEERVDKGETIPPQELTTKYA | S53 precursor |
| | VDPTAAGTLQTWLKKEGFTITGVTPDRTTIYASAPAS | [Bradyrhizobium |
| | QVEASLGVHTVRVTREGQTYTAASDVPSLPEDIGGA | canariense] |
| | VVNIGGLQPYRQARKHLRSYIQTTPEADGEEPAIANA | |
| | PPYLVPEILKAYDGARLGLTGKGQEIAILIDTVPLDTD | |
| | LTSFWTANGVAGSLARITKINVKGGALPTPSGEETLD | |
| | AEWASTIAPDANVRIYASGTLSFIDLDRALDRIYADA | |
| | LAQPKLRIVSISLGLSEAYMAKGEVDAEEARFVRFAA | |
| | LGVNVFVSTGDAGSNPGPDGHHANGPLAAEWMSTS | |
| | PHVVAVGGTSLRLANNGQVASETGWTGSGGGKSNF | |
| | QPRPAWQQGHGVPAGNQRMVPDVGAAADPNEGAL | |
| | VILNGQRLQYGGTSWSAPIWAGLCALINEARQNNHK | |
| | TPLPYLNSLIYPMIGSNCFRDELTGSNGAYSCGPGYD | |
| | LVTGIGSPDLKQLAAKLA | |
| 9 | MLRNERKIQSGFALGRMAAALVLTIGVHGVAMAVQ | Protein Peptidase |
| | TONIGASSWIATRTHAVNTAGATFNGNATDQEPVSI | S53 precursor |
| | AIALKLRNENALNNYIHELFRRGSPSYHHFLSAKDSV | [Burkholderia |
| | ATYAPTQQQAQAVADYLTRQGFTHVRIAPNRLVVS | singularis] |
| | ADGTVGAARNAFRTEIAHFTRAGHSGIANTTDIQIPA | |
| | SLSGQVDHVTGLQTLNRMQTFSLQHPSYTTLNSGAA | |
| | AYAPQEFATVYHAGNTPAGTGTTVALIGWGNMTNP | |
| | VNDLVQMEKSQNLAAVPTSIEPTSTDSSNDDSTQVE | |
| | WGMDAQAIVGISGGVKELIFYTSGGNYVQDPKTKS | |
| | WYSEGSYNSELLKAINLAVSDNKAKVINMSWGLAE | |
| | CRGDAGWADSAFKLGVTQGQTFVAATGDNGAYPC | |
| | EDSNHHAPANGSYSSNTELSVNYPASSPYVVAVGGT | |
| | TLNTSASDVYNSEASWPYSGGGISGEESTPTWQPSSY | |
| | AHRALPDLAFDADSTNSPILIYLTQSQTANISQSGFGY | |
| | AYGGTSLAAPLFVGAWARLETANKNGMGFAAPAIY | |
| | SYSTTFPFHDVTTGNNGYYSAAAGWDSATGWGSFDI | |
| | QAVRDFIANTPGFVSATNAPPN | |
| 10 | MKKTTSRVFKQGRLAAALAIAFAASGAALADATPGT | Protein Peptidase |
| | DASNWVPTRNQVHANATNAQLVGTANDSEPVSITV | S53 precursor |
| | TMKLRNESKLDQFIKEVHRPGSASFHKFLSASESTAD | [Burkholderia |
| | YAPTQQEAQAVADHLTRAGFTNVQIAPNRMAVTAQ | singularis] |
| | GTVGAARAAFHTSIGHFNVHGHDAIANTAQVEVPAA | |
| | LSGTVDRVLGLQTLHRPQVFSRTPKPAYTVNSSGGH | |
| | AYYPKEFATVYNAGSVPAATSTDVAIVGWGTMTNS | |
| | AADLAYMTKDQGLPSIPTTIKGYGGPTAGATGNDDS | |
| | GRGEWSMDAQAIAGVSGGVKSLTFYTAYSNIDSRHP | |
| | YGSATNAGIAAAINGAVSDNIAKVINMSWGDTNSEV | |
| | CSGTSDTAWLDSYFKLGVAQGQTFSVSSGDNGAYPC | |
| | NAPQNGAYGDKTQPTVSYPATSPYVVSVGGTTLNTT | |
| | TTDSYISETVWPYSGGGLSKIEAKPAWQTTVSGNYR | |
| | AVPDMVFDADWNNSPIRYYMAYVPDSSTSKGGGQT | |
| | PTQSGYYDNGGTSLASPLFVGSWARLESAHGNSMGF | |
| | AAPALYAYGTTMPVHDVTSGSNGLYTAGASWDNAS | |
| | GWGSFDISGMSTFITNTPGFVTTSNQH | |
| 11 | MSVRTDLAVEAKEIYEEKNAGEIPGVELKEYRQGRI | Protein Peptidase |
| | KVTEVNVLNEQGEKAMNKAIGNYITLEIPDINQYDT | S53 |
| | QYKEHISKILAKTLMPLLKIDDSMTALVVGLGNWNI | precursor |
| | TPDALGPKVIEKIMITRHLKEYIPNEIDEGIRPVCGISP | [Clostridium |
| | GVLGITGIETAEIIKAVSNKIKPDIILCIDALASRRLER | tetani] |
| | VNRTIQIGNTGISPGAGVGNRRMELNEKTLGVPVIAIG | |
| | VPTVVDAATVANDTIDMVLDEMIKVADKDKNFYN | |
| | MLKSLDRDEKERMIKEVLNPYVGELMVTPKDVDTL | |
| | MDSISKIISTGINIALQPALELEDINSYLN | |
| 12 | MANGKSTSPASQWVPLPGSNRQLLPQSVPIGPADLK | Protein Peptidase |
| | ATVALTVKVRSRGKLAELDDAVKKESAKPLKERTYI | S53 precursor |
| | SREELAQRYGADADDLDKVELYANKHHLRVADRDE | [Variovorax sp. |
| | ATRRVVLKGTLEDALSAFHADVHMYQHASGPYRGR | HW608] |
| | RGEILVPAELKDVVTGIFGFDTHPKHRAPRRLMGTSS | |
| | GTATNLGEFASEFATRYQFPTSSSSTKLDGTGQCIALI | |
| | ELGGGYSNNDLKIFFSEAGVPMPKVVAVSIDHGANH | |
| | PTPQGLADGEVMLDIEVAGVVAPGAKLAVYFAPNS | |
| | DSGFQDAIRAAVHDGARKPSVVSISWGEPDDELTAQ | |
| | SVQSYHEIFTEAAALGVTVCAASGDHGVADLDALH | |
| | WDKRIHVNHPSSDPLVLCCGGTQIDKNVDVVWNDG | |
| | TPFDPQVFGGGGWASGGGISPVFGVPDYQKGLPMPS | |
| | SLSTSQPGRGCPDIAMTADNYRTRVHGVDGPSGGTS | |
| | AVTPLMACLVARLNQAFEKNLGFVNPLLYANAQAF | |
| | TDITQGTNGINQTIEGYPAGKGWDACTGLGAPIGTVL | |
| | LQALGK | |
| 13 | MMKSSAAKQTVLCLNRYAVVALPLAIASFAAFGASP | Pseudomonapepsi |
| | ASTLWAPTDTKAFVTPAQVEARSAAPLLELAAGETA | n precursor |
| | HIVVSLKLRDEAQLKQLAQAVNQPGNAQFGKFLKR | [Pseudomonas sp. |
| | RQFLSQFAPTEAQVQAVVAHLRKNGFVNIHVVPNRL | 101] |
| | LISADGSAGAVKAAFNTPLVRYQLNGKAGYANTAP | |
| | AQVPQDLGEIVGSVLGLQNVTRAHPMLKVGERSAA | |
| | KTLAAGTAKGHNPTEFPTIYDASSAPTAANTTVGIITI | |
| | GGVSQTLQDLQQFTSANGLASVNTQTIQTGSSNGDY | |
| | SDDQQGQGEWDLDSQSIVGSAGGAVQQLLFYMADQ | |
| | SASGNTGLTQAFNQAVSDNVAKVINVSLGWCEADA | |
| | NADGTLQAEDRIFATAAAQGQTFSVSSGDEGVYECN | |
| | NRGYPDGSTYSVSWPASSPNVIAVGGTTLYTTSAGA | |
| | YSNETVWNEGLDSNGKLWATGGGYSVYESKPSWQS | |
| | VVSGTPGRRLLPDISFDAAQGTGALIYNYGQLQQIGG | |
| | TSLASPIFVGLWARLQSANSNSLGFPAASFYSAISSTP | |
| | SLVHDVKSGNNGYGGYGYNAGTGWDYPTGWGSLD | |
| | IAKLSAYIRSNGFGH | |
| 14 | MSDMEKPWKEEEKREVLAGHARRQAPQAVDKGPV | Protein |
| | TGDQRISVTVVLRRQRGDELEAHVERQAALAPHARV | Kumamolisin |
| | HLEREAFAASHGASLDDFAEIRKFAEAHGLTLDRAH | precursor [Bacillus |
| | VAAGTAVLSGPVDAVNQAFGVELRHFDHPDGSYRS | sp. MN-32] |
| | YVGDVRVPASIAPLIEAVLGLDTRPVARPHFRLRRRA | |
| | EGEFEARSQSAAPTAYTPLDVAQAYQFPEGLDGQGQ | |
| | CIAIIELGGGYDETSLAQYFASLGVSAPQVVSVSVDG | |
| | ATNQPTGDPNGPDGEVELDIEVAGALAPGAKIAVYF | |
| | APNTDAGFLNAITTAVHDPTHKPSIVSISWGGPEDSW | |
| | APASIAAMNRAFLDAAALGVTVLAAAGDSGSTDGE | |
| | QDGLYHVDFPAASPYVLACGGTRLVASAGRIERETV | |
| | WNDGPDGGSTGGGVSRIFPLPSWQERANVPPSANPG | |
| | AGSGRGVPDVAGNADPATGYEVVIDGETTVIGGTSA | |
| | VAPLFAALVARINQKLGKPVGYLNPTLYQLPPEVFH | |
| | DITEGNNDIANRARIYQAGPGWDPCTGLGSPIGIRLL | |
| | QALLPSASQAQP | |
| 15 | MRHRFGLSILFLVLVSSAVAQVIVPPTSVRRPGERPG | Protein Peptidase |
| | TAHTNYRIYIGPWRFPSVDSPFPELAAAHGPAAGQTI | S53 |
| | PGYHPADIRAAYNVPPNLGTQAIAIVDAFDLPTSLND | precursor |
| | FNFFSAQFGLPTEPSGVATASTNRVFQVVYASGTKPA | [Fimbriimonas |
| | TNADWGGEIALDIEWAHAMAPNAKIYLIEADSDSLL | ginsengisoli Gsoil |
| | DLLAAVRVAATQLSNVRQISMSFGANEFTNESASDS | 348] |
| | TFLGTNKVFFASSGDASNLVSYPAASPNVVGVGGTR | |
| | LALSNGSVVSETAWSSAGGGPSSREPRPTYQNSVSG | |
| | VVGSARGTPDIAAIADPETGVAVYDSTPIPGTGVGWF | |
| | VVGGTSLACPVCAGITNARGYFTASSFSELTRLYGLA | |
| | GTSFFRDITSGTSGQFSARVGYDFVTGLGSLLGIFGPF | |
| | ATSPSSLSVVSGTAVAGVPSNMVAKDGHDYVVRSA | |
| | SPAGGGQVATVQGTFASHPPAKAVQFGASVTVTAM | |
| | RTSGTTTLKLFNQATSAFESVANLTLGTTNTTVTVPIP | |
| | NAPKYFASDGTTKFQLTTTGPGTTQIRFGVDQVLLTL | |
| | TPTG | |
| 16 | MPTFLLPGSEQTCPPGARCVGKADPSARFEVTLVVR | Protein Peptidase |
| | QPAQDAFARHLEALHDVTRRPPALTREAYAAQYSA | S53 precursor |
| | AADDFAAVEQFAASEGLQVVRRDAAQRTIVLSGTV | [Pandoraea |
| | AQFNHAFEIDLQKIEHEGKSYRGRVGPVHLPQHLKT | thiooxydans] |
| | VVDAVLGLEDLPLARTHFRLQPAARSAAGFTPLELA | |
| | SIYQFPAGAGKGQAIALIELGGGVKTSDLTTYFSQLG | |
| | VTPPQVTAVSVDQATNSPTGDPNGPDGEVTLDVEIT | |
| | GAIAPEAHIVLYFAPNTEAGFFNAVSAAVHDTTHRPT | |
| | VISISWGGPEAAWTRQSLDAFDRALQAAAAMGVTV | |
| | CAASGDSGSSGSPGNGSPQVDFPASSPHVLACGGTRL | |
| | HASANRRDAESVWNDGAGGGASGGGVSAAFALPS | |
| | WQEGLQVTAADGTSQALTQRGVPDVAGDASPASGY | |
| | DVVVDAQATIVGGTSAVAPLWAGLIARLNASLGKPL | |
| | GYLNPILYQHPGVLNDITQGDNGEFSAAPGWDACTG | |
| | LGSPNGQKIAGVA | |
| 17 | MVRHPLRGSERTIPEDARILGDAHPAEQIRALVQLRR | Protein Peptidase |
| | PNEAELDVRLSGFVHAHAAGTPSPTPLTREEWAAQF | S53 precursor |
| | GAATDDIDAVRTFAREHGLQVAEVNVAAATVMLEG | [Burkholderiaceae |
| | SVEQFCRAFDTHLHRVAHGGSEYRGRSGPLRLPESL | bacterium 26] |
| | QDVVVAVLGLDSRPQAAPHFRFVPLPTGSVEPGGIRP | |
| | ARAAPTASYTPVQLAQLYGFPQGDGAGQCIAFVELG | |
| | GGYREDDLRAYFQEVGMPMPTVTAIPVGQGANRPT | |
| | GDPSGPDGEVMLDLEVAGAAAPGATLAVYFTVNTD | |
| | AGFVQAINAAIHDTKLRPSVVSISWGAPESAWTPQA | |
| | MQAVNAALQSAATMGVTVCAASGDSGSSDGQPDR | |
| | VDHVDFPASSPYALACGGTSVRASGNRIAEETVWND | |
| | GARGGAGGGGVSTVFALPSWQQGLAAQQTGGDSVP | |
| | LARRGVPDVSADADPLTGYVVRVDGESGVVGGTSA | |
| | AAPLWAALIARINAIKGRPAGYLHARLYQNPGAFND | |
| | IKQGNNGAFAAAPGWDACTGLGSPKGDAIANLF | |
| 18 | MTRHPVSDSGASNEHPVPAGAQCMGACDPAEHFNV | Protein Peptidase |
| | VVIVRRQSERAFRELVERIATGAPGAQPISREQYEQR | S53 precursor |
| | FSADAADVARVEAFAKTHGLVVVKADRDTRRVVLS | [Paraburkholderia |
| | GTVQQYNAAFGVDLQRFEHQVGKLKQHFRQPTGPV | sacchari] |
| | HLPEDLHEVITAVVGLDSRAKVQPHFRIDSQTPATPP | |
| | EKASQPGDGVVHAPIRAARAVSRSFTPLQLAELYDFP | |
| | PGDGKGQCIALIEMGGGYAQSDLDAYFSALGVTRPR | |
| | VEAVSVDQATNAPSGDPNGPDAEVTLDVEIAGALAP | |
| | GALIAVYFAPNSEAGFVDAVSAALHDSQRKAAIISIS | |
| | WGAPESIWSQQTLGALNDALQTAVALGVTVCCASG | |
| | DSGSSDGVTDGADHVDFPASSPYALGCGGTQLTAAN | |
| | GRITRETVWGSGANGATGGGVSATFAVPAWQKGLK | |
| | VSRGSGAARALALARRGVPDVAADADPATGYEVHI | |
| | GGMDTVVGGTSAVAPLWAALVARINAGSGKAAGFI | |
| | NAKLYARPGAFNDITSGSNGDYAARPGWDACTGLG | |
| | TPVGTRVAAAIGSA | |
| 19 | MADDSSPTTAADRPTLPGSARRPVAAAQAAGPLDD | Protein Peptidase |
| | AAPLEVTLVLRRRTALPAGTGRPAPMGRAEFAETHG | S53 precursor |
| | ADPADAETVTAALTAEGLRITAVDLPSRRVQVAGDV | [Modestobacter sp. |
| | ATFSRVFGVSLSRVESPDPVADRLVPHRQRSGDLAVP | DSM 44400] |
| | APLAGVVTAVLGLDDRPQARALFRPAAAVDTTFTPL | |
| | ELGRVYRFPSGTDGRGORLAILELGGGYTQADLDAY | |
| | WTTIGLADPPTVTAVGVDGAANAPEGDPNGADGEV | |
| | LLDIEVAGALAPGADLVVYFAPNTDRGFLDALSTAV | |
| | HADPTPTAVSISWGQNEDEWTAQARTAMDEALADA | |
| | AALGVTVCAAAGDDGSTDNAPDGQAHVDFPASSPH | |
| | ALACGGTTLRADPDTGEVSSETVWFHGTGQGGTGG | |
| | GVSAVFAVPDWQDGVRVPGDADTGRHGRGVPDVS | |
| | ADADPSTGYQVRVDGTDAVFGGTSAVSPLWSALTC | |
| | RLAEALGQRPGLLQPLIYAGLSAGEVAAGFRDVTSG | |
| | SNGAYDAGPGWDPCTGLGVPDGEALLVRLRTALG | |
| 20 | MSEPVPAAARRTIPGSERPPVDTAAAARQAVPADTR | Protein Peptidase |
| | VEATVVLRRRAELPDGPGLLTPAELAERHGADPADV | S53 precursor |
| | ELVTRTLTGLGVEVTAVDAASRRLRVAGPAGVLAE | [Pseudonocardia |
| | AFGTSLAQVSTPDPSGAQVTHRYRAGALSVPAELDG | sp. 73-21] |
| | VVTAVLGLDDRPQARARFRVATAAAASAGYTPIELG | |
| | RVYSFPEGSDGSGQTIAIIELGGGFAQSELDTYFAGLG | |
| | ISGPTVTAVGVDGGSNVAGRDPQGADGEVLLDIEVA | |
| | GALAPGADVVVYFAPNTDAGFLDAVAQAAHATPTP | |
| | AAISISWGGSEDTWTGQARTAFDAALADAAALGVTT | |
| | TVAAGDDGSTDRATDGKSHVDFPASSPHALACGGT | |
| | HLDANATTGAVTSEVVWNNGAGKGATGGGVSTVF | |
| | AQPSWQASAGVPDGPGGKPGRGVPDVSAVADPQTG | |
| | YRIRVDGQDLVIGGTSAVAPLWAALVARLVQAGRA | |
| | KLGLLQPKLYAAPTAFRDITEGDNGAYRAGPGWDA | |
| | CTGLGVPVGTALASALS | |

### REFERENCES

Each of the following references is hereby incorporated by reference in its entirety.
1. Benucci, llaria, et al. “A Minimally Invasive Approach for Preventing White Wine Protein Haze by Early Enzymatic Treatment.” Foods 11.15 (2022): 2246.
2. Van Sluyter, Steven C. et al. “Wine protein haze: Mechanisms of formation and advances in prevention.” loumal of agricultural and food chemistry 63.16 (2015): 4020-4030.
3. Van Sluyter, Steven C. et al. “Aspartic acid protease from Botrytis clr) erea removes hazeforming proteins during white winemaking.” Journal of agricultural and food chemistry 61.4D (2013): 9705-9711.
4. Theron, Louwrens Wiid, Marina Bely, and Benoit Divol. “Monitoring the impact of an aspartic protease (MpAPrl) on grape proteins and wine properties.” Applied microbiology and biotechnology 102 (2018): 5173-5 183.
5. Marangon, Matteo, et al. “Degradation of white wine haze proteins by Aspergillopepsin I and II during juice flash pasteurization.” Food Chemistry 135.3 (2012): 1157-1165.
6. Mutsaers, Johanna Henrica Gerdina Maria, Luppo Eden S and Wilbert Herman Marie Heijne. “Preparation of a stable beverage.” U.S. patent application Ser. No. 14/650,449.
7. Benucci, llaria, et al. “'Bromelain from pineapple stem in alcoholicacidic buffers for wine application.” Food Chemistry 124.4 (2011): 1349-1353.
8. Sun, Daqing, and Jack Harris. “Effective use of protease in winemaking.” U.S. patent application Ser. No. 10/085,323.
9. New Directions in Stabilization, Clarification, and Fining. In Managing Wine Quality.
10. Majewski, Peter, A. Barbalet, and E. Waters. “$1 billion hidden cost of bentonite fining.” Australian and New Zealand grapegrower and winemaker 569 (2011): 58-62.

### EXAMPLES

### Example 1: Dehazing Wine Using Pro-Kumamolisin

### Protease Production

The DNA sequence of an illustrative acid protease (SEQ ID NO: 1) was cloned into the expression vector pET29b(+) for protease production in E. coli. The completed DNA construct was transformed into an expression strain of E. coli (BL21) and grown at 37° C. in Terrific Broth using a baffled shake flask for 4-6 hours until the cell density (measured using OD 600) reached 0.6. The cultures were then induced with 0.5 mM of IPTG for protease expression. The culture was grown at 30° C. for 12 hours post induction before harvesting. The harvested cells were lysed using sonication and the protease (having the amino acid sequence of SEQ ID NO: 1) was purified from cell lysate using IMAC chromatography.

This method of producing a protease can be used for any other protease disclosed herein. More specifically, rather than expressing a DNA to produce a protein having the amino acid sequence of SEQ ID NO: 1, a nucleotide sequence can be expressed to produce a protein having the amino acid sequence of any one of SEQ ID NO: 2 to SEQ ID NO: 20, and variants thereof.

### Dehazing of White Wine

To demonstrate the efficacy of using an illustrative protease of the present disclosure, e.g., (having the amino acid sequence of SEQ ID NO: 1, in dehazing wine, a heat test was conducted using a newly fermented Sauvignon blanc white wine. The wine was filtered at 0.45 pm and its turbidity was measured. The wine sample was then heated in a water bath at 80° C. for 6 hours, cooled at 4° C. for 16 hours, and brought to room temperature for 2 hours before measuring its turbidity. The amount of haze was quantified by measuring the turbidity at OD600. As shown in FIG. 1, Control, the heat test demonstrated that the wine sample became hazy due to the presence of proteins naturally present in the wine. This haziness in untreated wine characterizes instability of wine over improper and/or long-term storage.

Notably, the proteases of the present disclosure do not require a heating step to boost enzymatic activity. In contrast, some proteases reported in literature require a heating step (e.g., 50° C. for 20 min) to boost the enzyme activity; this heating step may be harmful to a beverage of interest and is not preferred by wineries. Accordingly, the proteases of the present disclosure avoid the requirement for heating that may be harmful to a beverage of interest.

To avoid the negative effects of a long heating step, a protease of the present disclosure was used to dehaze a white wine sample. The protease treatment was applied to the wine sample during the winemaking process, with working temperatures spanning from near freezing temperatures to approximately room temperature. The treatment consisted of incubating the wine with the protease for a short period of time (less than 24 hours, depending on the amount of haze forming proteins naturally present in the wine sample), resulting in a heat-stable wine.

To validate the enzyme activity, a harsh heating step (80° C., 2 hours) is performed to simulate the effects of improper storage, but in an accelerated manner. So it's different process.

To further verify the enzymes can degrade haze forming proteins, such as thaumatin-like proteins (TLPs) and), chitinases (CHIs), and B-(l,3)glucanases which are present in grapes and are released during crushing and pressing, quantitative proteomic profiling and identification was conducted to compared the samples before and after the enzyme treatment.

FIG. 1 is a graph showing the effect of enzyme concentration on haze formation. The measurement of optical density of 600 nm (OD600) in the samples subjected to haze-inducing conditions is used to quantify the degree of haze formation within a given sample. The control is the white wine sample without the supplement of the protease.

As shown in FIG. 1, the serial dilution experiment indicates that when the wine samples were treated with 0.01 g/L were 0.1 g/L of protease at 18° C. for 18 hours, no haze is induced by the heat test (OD600=0). The control sample without the supplement of the protease can form visible haze precipitations with OD600>0.08.

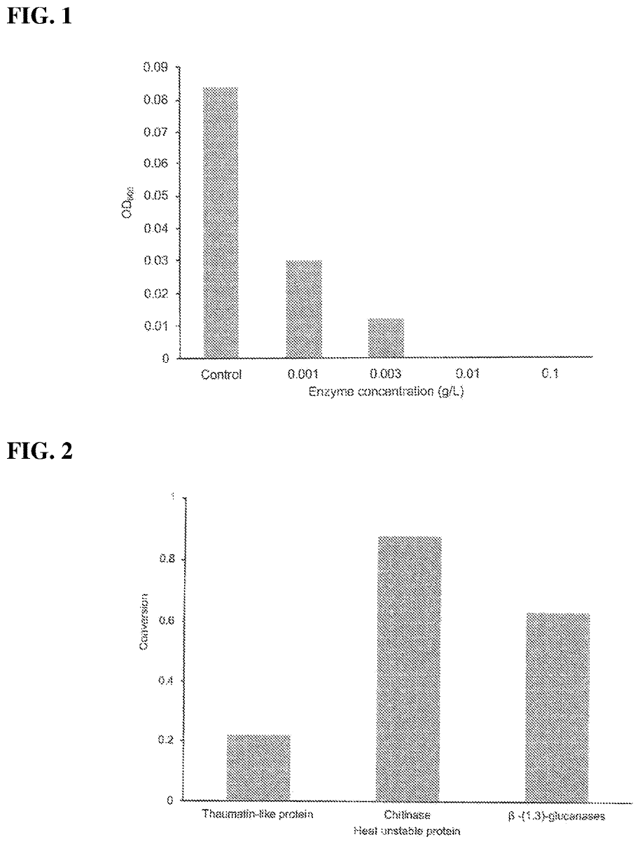

FIG. 2 is a graph showing the conversion rate of the major heat-induced haze proteins by protease treatment through the quantitative proteomic profiling. 0.01 g/L protease was used to treat wine at in 18° C. in 18 hours. The quantitative proteomic profiling and identification experiment is sequentially performed, and it validates the efficacy of the protease on three types of heat-unstable proteins (FIG. 2). Overall, the protease is able to dehaze the samples by degrading the haze-forming proteins.

### Example 2: Dehazing of White Wine Using S53 Protease Family Enzymes

### Enzyme Production

The DNA sequences encoding illustrative acid proteases of interest (SEQ ID NO: 1 to SEQ ID NO: 20) were cloned into the expression vector pET29b(+) for protease production in E. coli. The completed DNA construct was transformed into an expression strain of E. coli (BL21) and grown at 37° C. in Terrific Broth using a baffled shake flask for 4-6 hours until the cell density (measured using OD 600) reached 0.6. The cultures were then induced with 0.5 mM of IPTG for protease expression. The culture was grown at 30° C. for 12 hours post induction before harvesting. The harvested cells were lysed using sonication and the proteases having the amino acid sequence of one SEQ ID NO: 1 to SEQ ID NO: 20 were purified from cell lysate using IMAC chromatography.

### Dehazing of White Wine

To demonstrate the efficacy of using an illustrative proteases of the present disclosure, e.g., (having the amino acid sequence of SEQ ID NO: 1 to SEQ ID NO: 20) in dehazing wine, a heat test was conducted using a newly fermented Sauvignon blanc white wine. The wine was filtered at 0.45 pm and its turbidity was measured. The wine sample was then heated in a water bath at 80° C. for 6 hours, cooled at 4° C. for 16 hours, and brought to room temperature for 2 hours before measuring its turbidity. The heat test demonstrated that the wine sample became hazy due to the presence of proteins naturally present in the wine. The amount of haze was quantified by measuring the turbidity at OD600.

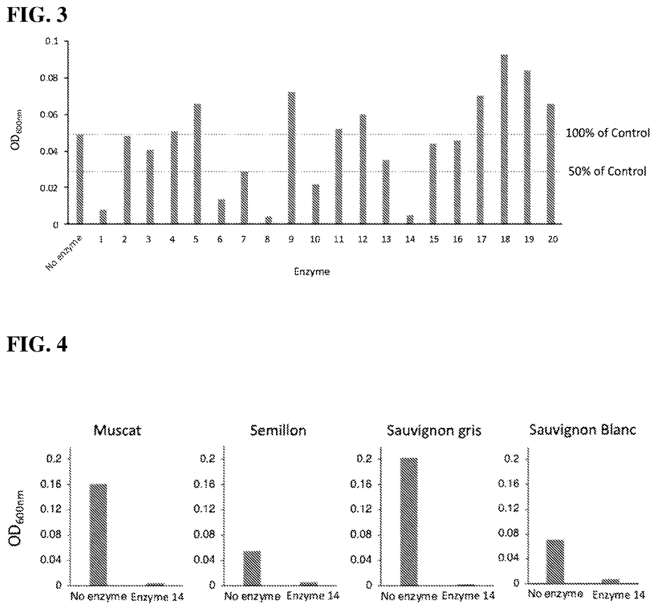

FIG. 3 demonstrates the efficacy of illustrative S53 proteases (of SEQ ID NO: 1 to SEQ ID NO: 20) in reducing wine haze and turbidity after heat test. Optical density measurements at 600 nm (OD600) indicate the level of turbidity in wine samples treated with various proteases from the S53 family. The results demonstrate the ability of several proteases within this enzyme family to effectively clarify wine by removing haze. Each data point represents the average turbidity reduction observed post-treatment, highlighting the potential of S53 proteases as a tool for improving wine clarity and quality.

The control wine sample (“No enzyme”), which had no added enzymes, showed a turbidity at OD600 of approximately 0.05. Enzymes represented by SEQ ID NO: 1, SEQ ID NO: 6, SEQ ID NO: 7, SEQ ID NO: 8, SEQ ID NO: 10, and SEQ ID NO: 14 achieved at least a 50% reduction in haze, and were therefore considered active. Among all the sequences, SEQ ID NO: 8 and SEQ ID NO: 14 performed the best, each achieving more than 90% haze removal.

### Example 3—Dehazing Wine Using Pro-Kumamolisin

A pro-Kumamolisin comprising an amino acid sequence of SEQ ID NO: 14 was obtained by the method described in Example 2.

FIG. 4 is a graph showing that Enzyme 14 of the present disclosure (i.e., having an amino acid sequence of SEQ ID NO: 14) is functional and capable of removing haze regardless of the wine varietal. Muscat, Semillon, Sauvignon Gris, and Sauvignon Blanc each exhibit distinct alcohol and sugar content profiles influenced by their wine styles and regions. Muscat wines vary in alcohol from 5% to 15% ABV and can range from dry to very sweet, impacting both alcohol and sugar levels. Semillon wines, used in both dry and renowned sweet styles like Sauternes, generally have alcohol content between 10% and 14% ABV, with sugar content varying significantly depending on the wine's sweetness. Sauvignon Gris typically has an alcohol content of about 11.5% to 13.5% ABV and is usually made in a dry style with low residual sugar. Lastly, Sauvignon Blanc is often found between 12% and 14% ABV and is predominantly produced as a dry wine with low sugar, although some late-harvest versions may be sweeter.

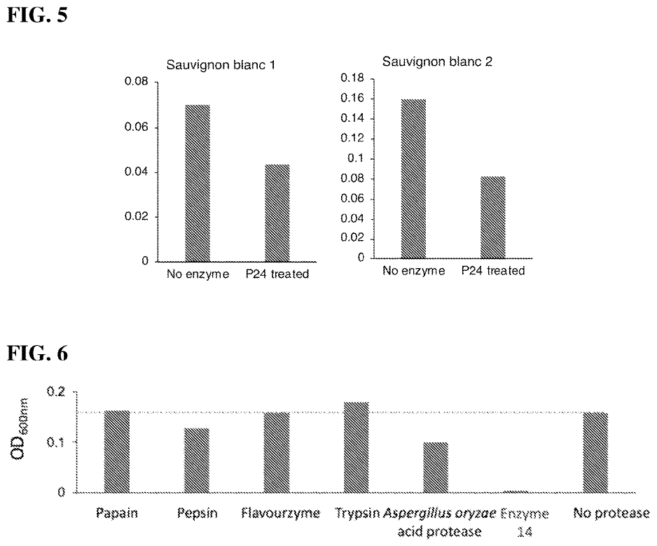

A protease of the present disclosure (e.g., having the amino acid sequence of SEQ ID NO: 14) was added to juice samples along with yeast strains selected for the fermentation process. The fermentation protocol used adhered to standard wine manufacturing practices, focusing on Sauvignon Blanc as the varietal of interest. Here, 0.05 g/L of protease was added to the juice samples. After fermentation, the wine samples underwent the heat cycles. FIG. 5 is a graph showing dehazing by a protease of the present disclosure of juice while fermenting into wine. The results, as depicted in FIG. 5, show that the addition of protease significantly reduced haze formation (here, the protease of SEQ ID NO: 14 is identified as “P24”). Specifically, the protease succeeded in removing approximately half of the haze in two distinct juice matrices, despite the absence of any optimization in enzyme concentration or treatment protocol. This outcome indicates that protease enzymes are a solution for improving the clarity and stability of wines.

Commercially available enzymes, sourced from Sigma Aldrich, along with an illustrative protease of the present disclosure, i.e., SEQ ID NO: 14, were evaluated under identical conditions using a third Sauvignon Blanc production lot. Each enzyme was added to the wine samples at a uniform concentration of 0.05 mg/mL and incubated at 18° C. for a 24-hour period. Following incubation, the samples underwent a heat stability test to assess their effectiveness in haze removal. FIG. 6 is a graph showing wine dehazing by the commercially-available proteases and by the illustrative protease of the present disclosure (e.g., having the amino acid sequence of SEQ ID NO: 14). According to the results displayed in FIG. 6, Protease (SEQ ID NO: 14) was the only enzyme that demonstrated complete efficacy in removing haze from the wine samples. None of the other enzymes tested could compete the potency of Protease (SEQ ID NO: 14) in achieving clear wine.

While preferred embodiments of the present disclosure have been shown and described herein, it will be obvious to those skilled in the art that such embodiments arc provided by way of example only. Numerous variations, changes, and substitutions will now occur to those skilled in the art without departing from the instant disclosure. It should be understood that various alternatives to the embodiments described herein may be employed in practicing the invention. It is intended that the following claims define the scope of the embodiments disclosed herein, and that methods and structures within the scope of these claims and their equivalents be covered thereby.

What is claimed is:

1. A method of dehazing a beverage of interest comprising a step of contacting a protease from the S53 family with the beverage of interest or adding the protease from the S53 family to the beverage of interest, thereby forming a composition comprising the beverage of interest and the protease.

2. The method of claim 1, wherein the S53 family protease comprises or consists of an amino acid sequence comprising at least 85% sequence identity to any one of SEQ ID NO: 1 to SEQ ID NO: 20.

3. The method of claim 2, wherein the S53 family protease comprises an active site comprising amino acid residues E266 or D266; F295, I295, or L295; S316; W317, L317, T317, or F317; G318; A349, S349; A350, S350, T350, or C350; G351; D352; S353, N353, A353, R353, E353, H353, or D353; G354; D367, S367, E367, or N367; G463; G464; T465; and/or S466; A467, L467, or W467 with respect to any one of SEQ ID NO: 1 to SEQ ID NO: 20.

4. The method of claim 1, wherein the S53 family protease comprises or consists of an amino acid sequence comprising at least 85% sequence identity to SEQ ID NO: 14.

5. The method of claim 4, wherein the S53 family protease comprises an active site having one or more amino acid substitutions selected from E266, D266, F295, I295, L295, S316, W317, L317, T317, F317, G318, A349, S349, A350, S350, T350, C350, G351, D352, S353, N353, A353, R353, E353, H353, D353, G354, D367, S367, E367, N367, G463, G464, T465, S466, A467, L467, and W467 with respect to SEQ ID NO: 14.

6. The method of claim 1, wherein the S53 family protease comprises an amino acid sequence of any one of SEQ ID NO: 1 to SEQ ID NO: 20.

7. The method of claim 6, wherein the S53 family protease comprises SEQ ID NO: 14.

8. The method of claim 1, wherein a protein load of the protease in the composition does not exceed 1%.

9. The method of claim 1, wherein the protease is added to the beverage of interest at a concentration ranging from about 0.001 mg/L to about 1000 mg/L.

10. The method of claim 1, wherein the beverage of interest is a wine selected from the group consisting of red wine, white wine, and rose wine.

11. The method of claim 1, wherein the method comprises incubating the composition for 30 minutes to 72 hours, for 3 days to 3 weeks, or for 3 months to 3 years.

12. The method of claim 1, wherein the protease is immobilized on a solid support prior to contacting with the beverage of interest.

13. The method of claim 12, wherein the solid support is a column or another device that permits the beverage to flow through and to contact the immobilized protease.

14. The method of claim 1, further comprising monitoring protein content or optical density of a sample of the beverage of interest or the composition to assess the degree of haze reduction.

15. The method of claim 1, wherein the beverage of interest is wine, juice, beer, cider, or an alcoholic or non-alcoholic beverage containing heat-unstable proteins.

16. The method of claim 1, wherein the beverage of interest comprises a juice selected from the group consisting of grape juice, apple juice, orange juice, berry juice, pineapple juice, pomegranate juice, plum juice, dandelion juice, rose hip juice, lychee juice, pear juice, and cherry juice.

17. The method of claim 1, wherein the beverage of interest comprises fermented grain.

18. A composition comprising a protease from the S53 family and a beverage of interest,
wherein the S53 family protease is selected from the group consisting of aspartic proteases, cysteine proteases, serine proteases, and metalloproteases,
wherein the S53 family protease comprises or consists of an amino acid sequence comprising at least 85%, at least 90%, at least 95%, or at least 98% sequence identity to any one of SEQ ID NO: 1 to SEQ ID NO: 20.

19. The composition of claim 18, wherein the beverage of interest is wine, juice, beer, cider, or an alcoholic or non-alcoholic beverage containing heat-unstable proteins.

20. A solid support comprising the composition of claim 18, wherein the S53 family protease is immobilized onto the solid support.
